(19) United States
(12) Patent Application Publication
(10) Pub. No.: US 2016/0244860 A1
(43) Pub. Date: Aug. 25, 2016

(54) ALIPHATIC-AROMATIC HETEROCYCLIC COMPOUNDS AND USES THEREOF IN METAL EXTRACTANT COMPOSITIONS

(71) Applicants: CYTEC INDUSTRIES INC., WOODLAND PARK, NJ (US); THE UNIVERSITY COURT OF THE UNIVERSITY OF EDINBURGH, EDINBURGH (GB)

(72) Inventors: James William ROEBUCK, Alloa (GB); Thomas SASSI, Stamford, CT (US); Adam James FISCHMANN, Long Island City, NY (US); Violina GRIFFIN, Norwalk, CT (US); Peter Anthony TASKER, Edinburgh (GB)

(21) Appl. No.: 15/048,034

(22) Filed: Feb. 19, 2016

(30) Foreign Application Priority Data
Feb. 20, 2015 (EP) EP15155983.8

(51) Int. Cl. C22B 3/16; C07D 401/14; C22B 3/00; C07D 413/04; C07D 403/14; C07D 231/12; C07D 401/04; C07D 403/04

(52) U.S. Cl. CPC C22B 3/1666; C07D 401/04; C07D 401/14; C07D 403/04; C07D 413/04; C07D 403/14; C07D 231/12; C22B 23/0415

(57) ABSTRACT

The invention relates to an aliphatic-aromatic heterocyclic compound A which comprises at least two heterocyclic rings B and C, to a metal extractant composition E comprising this aliphatic-aromatic heterocyclic compound A and an organic acid D having at least one carboxylic, sulfonic, sulfuric, phosphinic, phosphonic, or phosphoric acid group, and to a process to extract one or more metals M selected from the group consisting of Ni and Co from an aqueous acidic leach solution PL comprising ions of at least one of the metals M, and further, at least one kind of further ions selected from the group consisting of Fe ions, Al ions, Cu ions, Mg ions, Mn ions, and also silicate anions, by mixing the solution PL with an extractant composition E, separating the organic and aqueous phases, and recovering the metal M from the separated organic phase.

### CROSS-REFERENCE TO RELATED APPLICATIONS

This application claims benefit of priority of EP Application No. 15155983.8 filed Feb. 20, 2015, which is incorporated herein by reference in its entirety.

### FIELD OF THE INVENTION

This invention relates to aliphatic-aromatic heterocyclic compounds and metal extractant compositions containing same, processes for their preparation, and to uses thereof for extracting metal ions from aqueous solutions.

### BACKGROUND OF THE INVENTION

Hydrometallurgical methods to recover metals from ores, concentrates, scrap metals, scrap metal alloys, or intermediates have found good reception in separating value metals from accompanying materials, and also, in separating individual metals from their mixtures. There is a constant need for improving the selectivity and efficiency of extractant compositions for these purposes.

In many cases, acidic sulfate solutions are the primary materials used in these extraction processes. Leach solutions can be obtained by alkaline or acid leaching of ores or concentrates containing oxides or other compounds of base metals, or from scrap metals or scrap metal alloys.

In acid leaching processes, sulfuric acid is preferably used, thus providing aqueous sulfate solutions, which may also contain other anions such as chloride and nitrate, of metal ions such as Cu, Ni, Co, Zn, Cd, Pb, Mn, Al, Mg and Fe. Metals which are separated from these solutions, and further processed to yield the pure metal are usually referred to as “value metals” or “metal values”. Concentrations of these value metals in the aqueous solutions of their ions vary largely depending on the primary material used in the leaching process.

Nickel is mostly won from two types of ores, laterites and sulfides, and is usually accompanied by cobalt in these ores. Separation of nickel and cobalt from laterite ores is usually made by treatment with hot sulfuric acid at temperatures of about 250° C., and pressures of about 5 MPa (40 bar), known as the “High Pressure Acid Leaching” process. Historically, nickel and cobalt have been recovered from these acid leach solutions by precipitation as mixed sulfides, or mixed hydroxides, which processes are still used in older installations, and also newer plants which are designed to work without solvent extraction.

Although separation of nickel and cobalt can readily be effected with a solvent extraction (“SX”) reagent such as bis(2,4,4-trimethylpentyl)-phosphinic acid (commercially available as CYANEX® 272 from Cytec Industries Inc., Woodland Park, N.J. USA) where high separation factors can be reached in a pH region between 6.0 and 5.5, higher selectivity and purity of the resultant metal solutions are desired, as well as the ability to selectively extract Ni in a more acidic pH range.

In U.S. Pat. No. 4,254,087, to Grinstead, an extraction process has been described for recovering a first value metal which can be cobalt, copper, or nickel, from an acidic aqueous leach solution which further contains one or more second value metals selected from the group consisting of aluminium, calcium, iron, magnesium, and manganese, in which process an extractant composition is used which comprises a high molar mass alkylaromatic sulfonic acid, and a chelating amine which is a base having a pKₐ value (negative decadic logarithm of the acidity constant Kₐ) of from 3 to 9. The chelating amines disclosed therein have at least two coordination centres in their molecule, such as nitrogen, oxygen, or sulfur atoms, whereof at least one is a nitrogen atom incorporated in a ring referred to in that patent document as “semi-aromatic or aromatic” ring. Among the compounds disclosed, there are 2-picolylamines, 8-aminoquinoline, benzoxazoles and benzodiazoles, picolinic acid esters and amides, pyridylimidazoles, pyridylimidazolines, pyridyl tetrahydropyrimidines, and oxazoles. No chelating amines are disclosed that comprise at least two heteroatoms that are directly connected to each other.

In U.S. Pat. No. 4,356,309, to Lalk and Hamburg, N-substituted 2-(2-pyridyl)imidazoles have been disclosed where the substituent is chosen from C₁- to C₂₀-alkyl-substituted benzyl, or C₁₂- to C₂₀-alkyl which can optionally bear one or two —OR² radicals where R² can be hydrogen, or —CO—R³, R³ being C₁- to C₂₀-alkyl. These compounds are useful in metallurgical extractant systems as described in U.S. Pat. No. 4,254,087. No chelating amines are disclosed that comprise at least two heteroatoms that are directly connected to each other.

In U.S. Pat. No. 4,382,872, to Grinstead, which is based on a divisional application to the U.S. Pat. No. 4,254,087 mentioned hereinabove, an extractant composition is disclosed which comprises a high molar mass alkylaromatic sulfonic acid, and a chelating amine which is a strong base having a pKₐ value (negative decadic logarithm of the acidity constant Kₐ) of from 3 to 9. The chelating amines disclosed therein have at least two coordination centres in their molecule, such as nitrogen, oxygen, or sulfur atoms, whereof at least one is a nitrogen atom incorporated in a semi-aromatic or aromatic ring. Among the compounds disclosed, there are 2-picolylamines, 8-aminoquinoline, benzoxazoles and benzodiazoles, picolinic acid esters and amides, pyridylimidazoles, pyridylimidazolines, pyridyl tetrahydropyrimidines, and oxazoles. No chelating amines are disclosed that comprise at least two heteroatoms that are directly connected to each other.

In “Novel Solvent Extractants for Cobalt and Nickel” by B. Pesic and T. Zhou, Idaho National Engineering Laboratory 1996, tridentate molecules have been described as extractants for cobalt and nickel from acidic iron-bearing solutions. Two novel compounds have been described therein, 2,6-bis-(3-heptyl pyrazol-5′-yl)pyridine, and 2,6-bis-(3-nonyl pyrazol-5′-yl)pyridine. It has been found in the investigations upon which the present invention is based that these three-ring molecules show low solubility in the commonly used solvents in extraction processes such as hydrocarbon mixtures comprising mass fractions of from 4% to 23% of alkyl aromatics, 42% of cydoparaffins, and from 34% to 53% of linear and branched paraffins sold by Chevron Phillips Chemical Company LP of The Woodlands, Tex., USA under the trade mark ORFOM® SX-12, or that solutions of these extractants in the said commonly used solvents have elevated viscosity which makes such solutions unsuitable for solvent extraction processes due to impaired mixing and long phase disengagement times.

Combinations of N-alkyl-2-(2′-pyridyl) imidazole with dinonylnaphthalene sulfonic acid as described in “The Separation of Nickel (II) from Base Metal Ions using N-Alkyl-2-(2′-pyridyl) imidazole as Extractant in a Highly Acidic Sulfate Medium” (Hydrometallurgy vol. 121 to 124, 2012, pages 81 to 89) lead to third phase formation when trying to separate the loaded organic phase from the depleted aqueous acidic leach solution, necessitating the addition of long chain aliphatic alcohol to prevent such third phase formation.

An extractant composition is desired which selectively rejects undesired impurities present in the aqueous leach solutions including compounds of manganese, lead, alkaline earth metals, alkali metals and ammonium ions, selectively extracts certain metals in the form of their complexes by direct extraction or by differential stripping or by a combination of these, and allows to selectively remove individual base metals by differential extraction or by differential stripping.

Accordingly, metal extractant compositions suitable for use as solvent extractants to treat aqueous acidic sulfate pregnant leach solutions, which have a low viscosity and mix easily with the aqueous phase during the extraction step, and which also exhibit fast phase separation would be advantageous. Moreover, constituents of the extractants that remain chemically stable under the extraction process conditions, and that show appropriate complex formation with the desired metal ions, as well as easy isolation of the extracted metal ions from the organic phase after separation from the depleted aqueous leach solution, would be a useful advance in the art and could find rapid acceptance in the industry.

### SUMMARY OF THE INVENTION

It has been found that aliphatic-aromatic heterocyclic compounds comprising at least two, preferably exactly two, heterocyclic rings each having preferably from five to seven ring atoms, particularly preferably five or six ring atoms, and at least one alkyl substituent attached to one of the heterocyclic rings, can advantageously be used in the solvent extraction of metals from aqueous leach solutions. These leach solutions are obtained by treating ores or ore concentrates with dilute acid or alkali, and separating the aqueous phase rich in dissolved metal from the remaining solids. In the extraction step, these aqueous leach solutions are mixed with an organic phase which is an extractant solution comprising complexing agents which form chelates with the metals, which chelates are extracted into the organic phase. Aqueous and organic phases are then separated, and the loaded organic phase is treated in the so-called stripping step with an aqueous solution set to a pH where the partition equilibrium of the metal to be recovered is shifted so that at least a part of the metal is re-extracted into the aqueous phase in the form of hydrated metal ions. The depleted organic phase is recycled to be used in the extraction step again, and the aqueous strip solution is treated to recover the metal, commonly by electrowinning.

These and other objects, features and advantages of this invention will become apparent from the following summary of the invention and detailed description of the various embodiments of the invention taken in conjunction with the accompanying Examples and data tables.

In one aspect, the invention provides aliphatic-aromatic heterocyclic compound A of formula I which comprises two rings B and C, each ring being independently five-, six-, or seven-membered, preferably five- or six-membered, and each ring having at least one nitrogen atom as heteroatom in the ring structure, wherein at least one of the rings B and C has at least one further heteroatom selected from the group consisting of nitrogen, oxygen, and sulfur atoms, preferably nitrogen and oxygen atoms, and wherein at least two of the heteroatoms in at least one of the rings having two or more heteroatoms are directly attached to each other by a chemical bond, and wherein at least one of the rings B and C preferably bears a further substituent R which has from six to twenty-five carbon atoms,

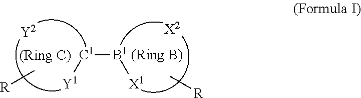

wherein in ring B, there is one heteroatom X1 which is directly bonded to ring atom B1 in ring B, and B1 is selected from the group consisting of a carbon atom and a nitrogen atom, and in ring C, there is one heteroatom Y1 which is directly bonded to ring atom C1 in ring C, and C1 is a carbon atom, and atoms B1 and C1 are connected to each other by a chemical bond which may be a single bond or a double bond, and wherein X2 and Y2 are each separately a sequence of from three atoms, in the case of a five-membered ring, to five atoms in the case of a seven-membered ring, and are selected from heteroatoms and carbon atoms. The substituent R attached to ring B, if different from a hydrogen atom, is referred to as RB, and the substituent R attached to ring C, if different from a hydrogen atom, is referred to as RC. Both RB and RC are referred to as R. If both rings B and C bear such substituent, RB may be the same as RC, or may be different from RC.

Preferably, at least one of the rings B and C bears a further substituent R which has from six to twenty-five carbon atoms, and which may optionally be substituted.

Further preferably, the substituent R is selected from the group consisting of a linear alkyl group, a branched alkyl group, a cyclic alkyl group, and an alkyl-substituted aryl group. In a preferred embodiment, the substituent R may contain heteroatoms which are preferably oxygen or nitrogen. It is also possible, in a preferred embodiment, that the atom of the substituent R directly bonded to either ring B or C is a heteroatom which is preferably oxygen or nitrogen. It is also possible that the substituent R contains one or more double bonds.

Preferably, the rings B and C are connected by a single bond.

In another embodiment, the rings B and C are connected by a double bond.

The heteroatoms in rings B and C are selected from the group consisting of nitrogen N, oxygen O, and sulfur S. Preferred are nitrogen and oxygen, and particularly preferred is that both rings contain only nitrogen atoms as heteroatoms, or that one ring contains only nitrogen atoms, and the other ring only contains nitrogen and oxygen atoms.

In one preferred embodiment, the ring B is a five-membered ring having two heteroatoms whereof one is a nitrogen atom, and one further heteroatom selected from the group consisting of nitrogen, oxygen, and sulfur atoms, preferably, from nitrogen and oxygen atoms, which two heteroatoms may be directly attached to each other by a chemical bond if both are nitrogen.

In another preferred embodiment, the ring B is a five-membered ring having three heteroatoms whereof two are nitrogen atoms, and one further heteroatom selected from the group consisting of nitrogen, oxygen, and sulfur atoms. Particularly preferred is a five-membered ring with two nitrogen atoms and one oxygen atom.

In another preferred embodiment, the ring B is a five-membered ring having four heteroatoms whereof all are nitrogen atoms.

In another preferred embodiment, the ring C has five or six atoms in the heterocyclic ring which is an aromatic ring, or a cycloaliphatic ring having at least one double carbon-carbon bond in the cycloaliphatic ring, and has a nitrogen atom in the 2-position relative to the atom connected to the ring B.

In another preferred embodiment, the ring B carries a substituent R which is linear or branched or cyclic alkyl group having from six to twenty-five carbon atoms.

In another preferred embodiment, the ring C is a pyrrole ring, a pyrazole ring, a triazole ring, a 1,3,5-triazine ring, a pyrazine ring, a pyrimidine, a pyridazine or a pyridine ring. In another preferred embodiment, the ring C carries more than one ring B attached to it. In this context, the ring C is preferably a triazine ring.

In another preferred embodiment, the ring B is selected from the group consisting of pyrazole, isoxazole, 1,3,4-oxadiazole, 1,2,4-oxadiazole, 1,2,3-triazole, 1,2,4-triazole and 1,2,3,4-tetrazole.

It is particularly preferred to combine these preferred embodiments, such as the preferred structures of ring B and number of atoms therein, with preferred structures of ring C and number of atoms therein, and the preferred substituents R, and the nature of the heteroatoms.

Accordingly, in one such combined embodiment the invention provides compounds A of Formula (II)

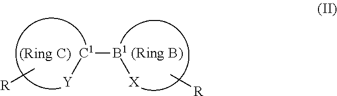

comprising an aliphatic-aromatic heterocyclic ring system of rings B and C, or salts or tautomers thereof, wherein
ring B is chosen from a pyrrole, pyrazole, isoxazole, 3-yl-oxadiazole, triazole, or tetrazole ring, wherein ring atom B¹ is a carbon or nitrogen atom and X is a nitrogen or oxygen heteroatom directly bonded to B¹;
ring C is chosen from a pyrazole, pyridine, pyridazine, pyrimidine, pyrazine, or triazine ring, wherein ring atom C¹ is a carbon atom and Y is a nitrogen atom directly bonded to C¹;
rings B and C are joined together by direct bond at atoms B¹ and C¹; and
R is present at one or more substitutable position of either or both of said rings and when present is chosen from (i) a C₆-C₂₅ linear or branched alkyl or alkenyl wherein from 1 to 3 carbon atoms is optionally substituted with a nitrogen or oxygen atom; or (ii) a cydohexyl wherein from 1 to 3 carbon atoms is optionally substituted with a nitrogen or oxygen atom; N-cyclohexyl; N,N-dicyclohexyl; a pyrazole; or phenyl any of which is optionally substituted with a C₁-C₆ linear or branched alkyl,
with the proviso that when R of ring C is a pyrazole, the pyrazole is nitrogen connected to ring C.

As those skilled in the art will be able to ascertain, other specific combinations of ring B and ring C are also contemplated from the disclosure, but are not specifically identified or listed herein.

A further aspect of the invention is a metal extractant composition E comprising an aliphatic-aromatic heterocyclic compound A as detailed hereinabove, and an organic acid D having at least one carboxylic, sulfonic, sulfuric, phosphonic, phosphinic, or phosphoric acid group, or salt thereof.

Preferably, the organic acid D has at least one carboxylic, sulfonic, sulfuric, phosphonic, phosphinic, or phosphoric acid group attached to an aromatic molecule, which forms the organic portion of the organic acid. A specific, non-limiting, example of such an organic acid is dinonyl naphthalene sulfonic acid (DNNSA).

In the same or another preferred embodiment, the organic acid D has at least one linear, branched, or cyclic alkyl substituent having from six to twenty-five carbon atoms on the aromatic molecule.

A preferred metal extractant composition E has an amount of substance-ratio of A and D of from 95 mol:5 mol to 5 mol:95 mol, more preferred, from 90 mol:10 mol to 30 mol:70 mol, especially preferred, from 80 mol:20 mol to 40 mol:60 mol, particularly preferred, from 70 mol:30 mol to 50 mol:50 mol, and most preferred, from 65 mol:35 mol to 55 mol:45 mol.

Preferably, the metal extractant composition E comprises a mixture of the heterocyclic compound A and the organic acid D dissolved in an organic solvent S which is not homogeneously miscible with water. Such solvents are preferably aliphatic hydrocarbons, and mixed aromatic-aliphatic hydrocarbons. A substance is regarded as being homo-geneously miscible with water for the purpose of this invention if there is no phase separation (=only one phase exists) in a temperature range of from 20° C. to 95° C. for mixtures comprising mass fractions of the substance under consideration from more than 0 kg/kg to less than 1 kg/kg, where a mass fraction is defined in the usual way as being the ratio of the mass of the substance under consideration to the mass of the mixture comprising this substance and water.

Yet another aspect of the invention includes processes to extract one or more metals M selected from the group consisting of Ni, Co, and Cu from an aqueous acidic leach solution PL comprising ions of at least one of the metals M, and further, at least one kind of further ions selected from the group consisting of Fe ions, Al ions, Mg ions, Mn ions, and also silicate anions, by
mixing the solution PL with an extractant composition E, until the following condition is fulfilled for the concentration c₁(M) of metal M in the aqueous phase after mixing during a time t1: [c₀(M)−c₁(M)]/[c₀(M)−cₑ(M)]>0.1, where c₀(M) is the concentration of metal M in the solution PL before mixing, and cₑ(M) is the equilibrium concentration of metal M in the aqueous phase after mixing and reaching the equilibrium,
separating the organic and aqueous phases, and
recovering the metal M from the separated organic phase by re-extraction with an aqueous phase which is neutral, acidic or alkaline, or by precipitation of the metal M by addition of a chemical compound that forms an insoluble compound with the said metal M.

### DETAILED DESCRIPTION OF THE PREFERRED EMBODIMENTS

The mention of metal M also includes the presence of more than one of the metals M. Therefore, if there are more than one metals M in any of the solutions mentioned herein, the term “concentration of metal M” means the “sum of the concentrations of all metals M” present in the said solutions. “Metals” herein is also short for “metal ions”.

In the following formulae, the branched nonyl group is always shown as one of the isomers, 1-methyl-1-ethylhexyl, but other branched isomers such as 1,1-dimethylheptyl are also present (or contemplated) in the isomer mixture.

Preferred heterocyclic compounds A which have been synthesised include: Compound 1: pyridine, 2-(5-[1-methyl-1-ethylhexyl]-1H-pyrazol-3-yl)-,

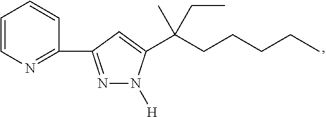 [SMILES: CCCCCC(C)(CC)c1cc(-c2ccccn2)n[nH]1]

wherein the alkyl chain can also have other branched C₉-alkyl isomers present;

Compound 2: pyridine, 2-(5-n-nonyl-1H-pyrazol-3-yl)-,

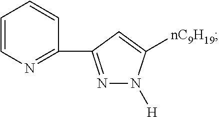

Compound 3: pyridine, 2-1H-pyrazol-1-yl-,

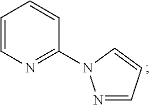 [SMILES: c1ccc(-n2cccn2)nc1]

Compound 5: pyrazine, 2-(5-[1-methyl-1-ethylhexyl]-1H-pyrazol-3-yl)-,

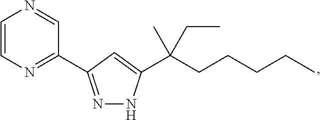 [SMILES: CCCCCC(C)(CC)c1cc(-c2cnccn2)n[nH]1]

wherein the alkyl chain can also have other branched C₉-alkyl isomers present;

Compound 6: 5-[1-methyl-1-ethylhexyl]-3-(2-pyridyl)isoxazole,

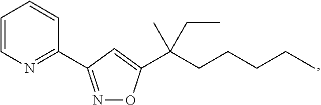 [SMILES: CCCCCC(C)(CC)c1cc(-c2ccccn2)no1]

wherein the alkyl chain can also have other branched C₉-alkyl isomers present;

Compound 7: 3-[1-methyl-1-ethylhexyl]-5-(2-pyridyl)isoxazole,

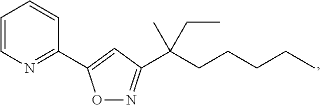 [SMILES: CCCCCC(C)(CC)c1cc(-c2ccccn2)on1]

wherein the alkyl chain can also have other branched C₉-alkyl isomers present;

Compound 8: pyridine, 2-(5-(1-butylheptyl)-1,2,4-oxadiazol-3-yl)-,

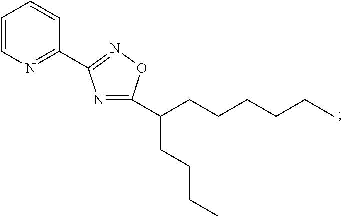 [SMILES: CCCCCCC(CCCC)c1nc(-c2ccccn2)no1]

Compound 9: pyridine, 2-(5-(2,4,4-trimethylpentyl)-1,2,4-oxadiazol-3-yl)-,

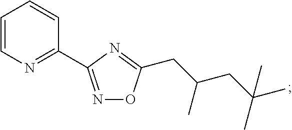 [SMILES: CC(Cc1nc(-c2ccccn2)no1)CC(C)(C)C]

Compound 10: pyridine, 2-(5-[1-methyl-1-ethylhexyl]-1,2,4-oxadiazol-3-yl)-,

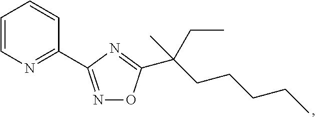 [SMILES: CCCCCC(C)(CC)c1nc(-c2ccccn2)no1]

wherein the alkyl chain can also have other branched C₉-alkyl isomers present;

Compound 11: pyridine, 2-(5-(1-(3-methylhexyl)-6-methylnon-1-yl)-1,2,4-oxadiazol-3-yl)-,

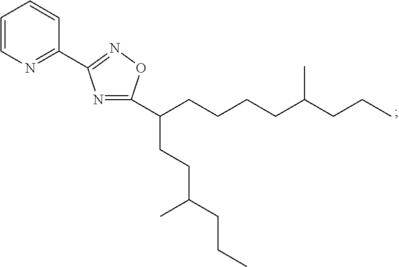 [SMILES: CCCC(C)CCCCC(CCC(C)CCC)c1nc(-c2ccccn2)no1]

Compound 12: pyridine, 2-(5-(heptadeca-8,11-dienl-yl))-1,2,4-oxadiazol-3-yl)-,

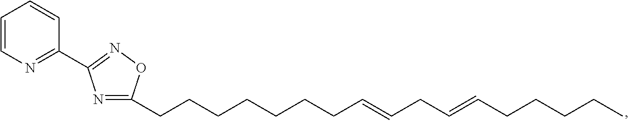 [SMILES: CCCCC/C=C/C/C=C/CCCCCCCc1nc(-c2ccccn2)no1]

where other fatty acid residues can also be present, due to the use of soybean fatty acid methyl ester in the synthesis of this compound;

Compound 13: pyridine, 2-(5-[1-methyl-1-ethylhexyl]-1,3,4-oxadiazol-2-yl)-,

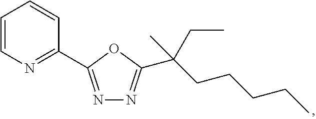 [SMILES: CCCCCC(C)(CC)c1nnc(-c2ccccn2)o1]

wherein the alkyl chain can also have other branched C₉-alkyl isomers present;

Compound 14: pyridine, 2-(5-(1-hexylnonyl)-1,3,4-oxadiazol-2-yl)-,

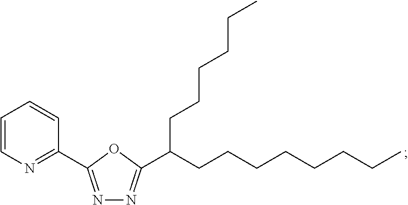 [SMILES: CCCCCCCCC(CCCCCC)c1nnc(-c2ccccn2)o1]

Compound 15: pyridine, 2-(5-[1-methyl-1-ethylhexyl]-1,2,4-triazol-3-yl)-,

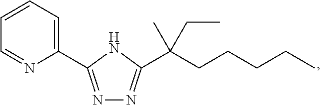 [SMILES: CCCCCC(C)(CC)c1nnc(-c2ccccn2)[nH]1]

wherein the alkyl chain can also have other branched C₉-alkyl isomers present;

Compound 16: pyridine, 2-(5-(1-butylheptyl)-1,2,4-triazol-3-yl)-,

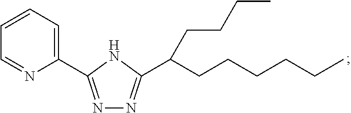 [SMILES: CCCCCCC(CCCC)c1nnc(-c2ccccn2)[nH]1]

Compound 17: pyridine, 2-(5-(2,4,4-trimethylpentyl)-1,2,4-triazol-3-yl)-,

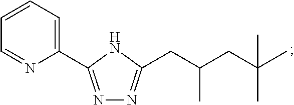 [SMILES: CC(Cc1nnc(-c2ccccn2)[nH]1)CC(C)(C)C]

Compound 18: a mixture of

pyridine, 2-(1-methyl-5-[1-methyl-1-ethylhexyl]-1,2,4-triazol-3-yl)-,
pyridine, 2-(2-methyl-5-[1-methyl-1-ethylhexyl]-1,2,4-triazol-3-yl)-, and
pyridine, 2-(3-methyl-5-[1-methyl-1-ethylhexyl]-1,2,4-triazol-3-yl)-
wherein the alkyl chain can also have other branched C₉-alkyl isomers present,

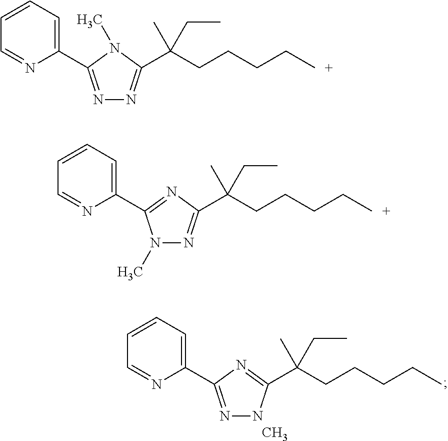

Compound 19a: pyridine, 2-(1-octadecyltetrazol-5-yl),

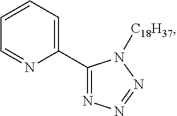

and

Compound 19b: pyridine, 2-(2-octadecyltetrazol-5-yl),

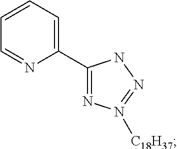

Compound 20: 1,3,5-triazine, 2,4-bis(2,4-dimethylphen-1-yl)-6-1H-pyrazol-1-yl-,

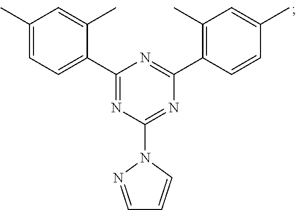

Compound 21: 1,3,5-Triazin-2-amine, 4-(4-morpholino)-6-1H-pyrazol-1-yl-N-cydohexyl,

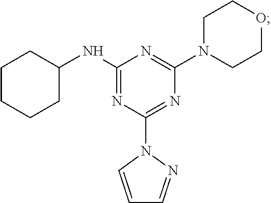 [SMILES: c1cnn(-c2nc(NC3CCCCC3)nc(N3CCOCC3)n2)c1]

Compound 22: 1,3,5-triazin-2,4-diamine-6-1H-pyrazol-1-yl-N²-[1-methyl-1-ethylhexyl]-N⁴,N⁴-dicyclohexyl,

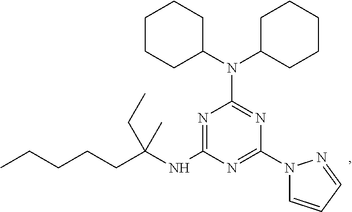 [SMILES: CCCCCC(C)(CC)Nc1nc(N(C2CCCCC2)C2CCCCC2)nc(-n2cccn2)n1]

wherein the alkyl chain can also have other branched C₉-alkyl isomers present;

Compound 23: 1,3,5-triazin-2,4-diamine-6-1H-pyrazol-1-yl-N2,N4-bis[1-methyl-1-ethylhexyl],

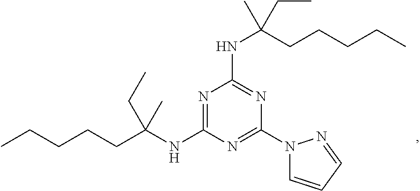 [SMILES: CCCCCC(C)(CC)Nc1nc(NC(C)(CC)CCCCC)nc(-n2cccn2)n1]

wherein the alkyl chains can also have other branched C₉-alkyl isomers present;

Compound 24: 1,3,5-triazin-2-amine, 4,6-di-1H-pyrazol-1-yl-N-[1-methyl-1-ethylhexyl],

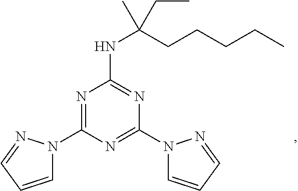 [SMILES: CCCCCC(C)(CC)Nc1nc(-n2cccn2)nc(-n2cccn2)n1]

wherein the alkyl chain can also have other branched C₉-alkyl isomers present;

Compound 25: 1,3,5-triazin-2-amine, N-methyl-N-butyl-4-1H-pyrazol-1-yl-6-(branched-decyloxy),

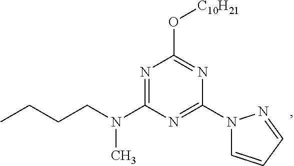

Compound 26: 5-nonyl-3-(5-nonylpyrazol-3-yl)pyrazole,

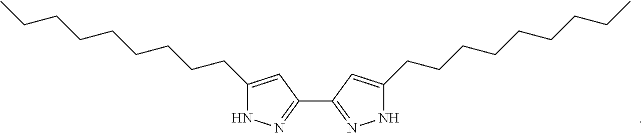 [SMILES: CCCCCCCCCc1cc(-c2cc(CCCCCCCCC)[nH]n2)n[nH]1]

The substituents R, which can be located at one or more substitutable position on either or both rings, are linear or branched alkyl groups having from six to twenty-five carbon atoms, preferably from seven to twenty-three, and particularly preferred, from eight to twenty-one carbon atoms in certain embodiments. They may also carry one or more olefinic unsaturations in the carbon chain. Substituent R can also include cyclic alkyl groups such as cydohexyl, N-cyclohexyl, N,N-dicyclohexyl. In certain embodiments, from 1 to 3 carbon atoms of the linear or branched alkyl groups or cyclic alkyl groups can be optionally substituted with a nitrogen or oxygen atom. In other embodiments, R can be aryl or heteroaryl such as or phenyl or pyrazole. Substitutent R can itself be optionally substituted with a linear or branched alkyl group from 1 to 25 carbon atoms, and preferably from 1 to 12 carbon atoms, more preferably from 1 to 6 carbon atoms; or cyclic alkyl from 6 to 12 carbon atoms.

As those skilled in the art will appreciate, the aliphatic-aromatic heterocyclic compounds A described herein and contemplated for use as metal extractants can also include their corresponding salts, which can be derived from various organic and inorganic acids and bases by procedures known in the art. Acceptable salt forms of the compounds A can be prepared by conventional and routine methods known to those skilled in the art. Tautomeric forms of the aliphatic-aromatic heterocyclic compounds A are also contemplated for use as metal extractants.

The main advantages realised with the extractant composition of the present invention are its chemical stability under the conditions used in extraction processes, the low aqueous solubility of the aliphatic-aromatic heterocyclic compound A according to the invention, and the possibility to remove copper from the organic phase generated during extraction by treatment of the organic phase, particularly with aqueous ammonia solution, after re-extraction of cobalt and nickel into the aqueous phase. Although the 1,3,4-oxadiazoles have a lower chemical stability than the other compounds mentioned, their low solubility in the aqueous phase, and their selectivity makes them useful for the purposes of this invention.

The heterocyclic compounds mentioned hereinabove all show good selective extraction of Ni from mixtures containing other metal salts, particularly Co, Cu, Fe, Al, Mg, and Mn salts. Particularly preferred heterocyclic compounds are those compounds A that have a ring C which is pyridine or pyrazole or 1,3,5-triazine, and a ring B that is an oxadiazole, a pyrazole or a triazole. Especially preferred are alkyl-2-(2-pyridyl)-1,2,4-oxadiazoles, as compounds 8, 9, 10, 11, and 12; alkyl-2-(2-pyridyl)pyrazoles, as compounds 1 and 2; bis-pyrazoles such as compound 26; alkyl-3-(2-pyridyl)-1,2,4-triazoles, as compounds 15, 16, 17, 18; tetrazoles as compound 19; and N-(2-pyridyl)-pyrazoles, as compound 3, and 1,3,5-triazine-pyrazoles, viz., compounds 22, 23, 24, and 25.

In accordance with the above, the invention includes at least the following embodiments:

### Embodiment 1

An aliphatic-aromatic heterocyclic compound A of formula I, or salts or tautomers thereof, which comprises two rings B and C, each ring being independently five- or six-membered, and each ring having at least one nitrogen atom as heteroatom in the ring structure, wherein at least one of the rings B and C has at least one further heteroatom selected from the group consisting of nitrogen atoms and of oxygen atoms, and wherein at least two of the heteroatoms in at least one of the rings having two or more heteroatoms are directly attached to each other by a chemical bond, and wherein at least one of the rings B and C bears a further substituent R at one or more substitutable position which substituent R has from six to twenty-five carbon atoms which are optionally substituted, and wherein from 1 to 3 carbon atoms is optionally substituted with a nitrogen or oxygen atom

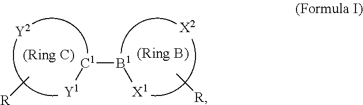

wherein in ring B, there is one heteroatom X1 which is directly bonded to ring atom B1 in ring B, and B1 is selected from the group consisting of a carbon atom and a nitrogen atom, and in ring C, there is one heteroatom Y1 which is directly bonded to ring atom C1 in ring C, and C1 is a carbon atom, and atoms B1 and C1 are connected to each other by a chemical bond which may be a single bond or a double bond, and wherein X2 and Y2 are each separately a sequence of from three atoms, in the case of a five-membered ring, to four atoms in the case of a six-membered ring, and are selected from heteroatoms and carbon atoms.

### Embodiment 2

The aliphatic-aromatic heterocyclic compound A of embodiment 1, wherein ring B has five ring atoms, and ring C has five or six ring atoms.

### Embodiment 3

The aliphatic-aromatic heterocyclic compound A of embodiment 1 or of embodiment 2, wherein the substituent R is selected from the group consisting of a linear alkyl group, a branched alkyl group, a cyclic alkyl group, and an alkyl-substituted aryl group.

### Embodiment 4

The aliphatic-aromatic heterocyclic compound A of embodiment 1 or of embodiment 3, wherein the ring B is a five-membered ring having two heteroatoms whereof one is a nitrogen atom, and one further heteroatom selected from the group consisting of nitrogen and oxygen atoms, which two heteroatoms are directly attached to each other by a chemical bond.

Embodiment 5 The aliphatic-aromatic heterocyclic compound A of embodiment 1 or of embodiment 3, wherein the ring B is a five-membered ring having three heteroatoms whereof two are nitrogen atoms, and one further heteroatom selected from the group consisting of nitrogen atoms and of oxygen atoms.

### Embodiment 6

The aliphatic-aromatic heterocyclic compound A of embodiment 1 or of embodiment 3, wherein the ring B is a five-membered ring having four heteroatoms whereof all four are nitrogen atoms.

### Embodiment 7

The aliphatic-aromatic heterocyclic compound A of any of embodiments 4, 5, and 6, wherein the ring C has five or six atoms in the heterocyclic ring which is either an aromatic ring, or a cycloaliphatic ring having at least one double carbon-carbon bond in the cycloaliphatic ring, and has a nitrogen atom in the 2-position relative to the atom connected to the ring B.

### Embodiment 8

The aliphatic-aromatic heterocyclic compound A of any of embodiments 4, 5, and 6, wherein the ring B carries a substituent R which is linear or branched or cyclic alkyl group having from six to twenty-five carbon atoms.

### Embodiment 9

The aliphatic-aromatic heterocyclic compound A of embodiment 7, wherein the ring C is a pyrrole ring, a pyrazole ring, a triazole ring, a 1,3,5-triazine ring, a pyrazine ring, a pyrimidine, a pyridazine or a pyridine ring.

### Embodiment 10

The aliphatic-aromatic heterocyclic compound A of any of embodiments 1 to 3, wherein the ring B is selected from the group consisting of pyrazole, isoxazole, 1,3,4-oxadiazole, 1,2,4-oxadiazole, 1,2,4-triazole, 1,2,3-triazole, and 1,2,3,4-tetrazole.

### Embodiment 11

The aliphatic-aromatic heterocyclic compound A of any of embodiments 8 to 10, wherein the ring C carries substituent R at one or more substitutable positions on the ring which is chosen from i) a linear or branched alkyl or alkenyl having from six to twenty-five carbon atoms wherein from one to three carbon atoms are optionally substituted with a nitrogen or oxygen atom; or ii) a cydohexyl wherein from one to three carbon atoms are optionally substituted with a nitrogen or oxygen atom; N-cydohexyl; N,N-dicylcohexyl; pyrazole; or phenyl, any of which is optionally substituted with a linear or branched alkyl having from one to six carbon atoms.

### Embodiment 12

The aliphatic-aromatic heterocyclic compound A of any of embodiments 1 to 11, wherein compound A is chosen from Compound 1, 2, 3, 5, 6, 7, 8, 9, 10, 11, 12, 13, 14, 15, 16, 17, 19a, 19b, 20, 21, 22, 23, 24, 25, or 26; or from mixed isomers of pyridine, 2-(methyl)-5-branched-nonyl-1,2,4-triazole-3-yl) (Compound 18); or from salts or tautomers of any of these Compounds.

### Embodiment 13

The aliphatic-aromatic heterocyclic compound A of embodiment 12, wherein compound A is chosen from Compound 1, Compound 8, or Compound 23.

### Embodiment 14

A metal extractant composition E comprising an aliphatic-aromatic heterocyclic compound A of any of embodiments 1 to 13, and an organic acid D having at least one carboxylic, sulfonic, sulfuric, phosphinic, phosphonic, or phosphoric acid group, or salt thereof.

### Embodiment 15

The metal extractant composition E of embodiment 14, wherein the organic acid D has at least one carboxylic, sulfonic, sulfuric, phosphinic, phosphonic, or phosphoric acid group attached to an aromatic molecule.

### Embodiment 16

The metal extractant composition E of embodiment 15, wherein the organic acid D has at least one linear, branched, or cyclic alkyl substituent having from six to twenty-five carbon atoms on the aromatic molecule.

### Embodiment 17

The metal extractant composition E according to any of embodiments 14 to 16, wherein the amount of substance-ratio of A and D is from 95 mol:5 mol to 5 mol:95 mol.

### Embodiment 18

The metal extractant composition E according to any of embodiments 14 to 17, wherein the mixture of the heterocyclic compound A and the organic acid D is dissolved in an organic solvent S which is not homogeneously miscible with water.

### Embodiment 19

The metal extractant composition E according to embodiment 18, wherein the organic solvent S is selected from the group consisting of aliphatic hydrocarbons, and mixed aromatic-aliphatic hydrocarbons.

### Embodiment 20

A process to extract one or more metals M selected from the group consisting of Ni and Co from an aqueous acidic leach solution PL comprising ions of at least one of the metals M, and further, at least one kind of further ions selected from the group consisting of Fe ions, Al ions, Cu ions, Mg ions, Mn ions, and also silicate anions, by
mixing the solution PL with an extractant composition E according to any of embodiments 14 to 19, until the following condition is fulfilled for the concentration c₁(M) of metal M in the aqueous phase after mixing during a time t1: [c₀(M)−c₁(M)]/[c₀(M)−cₑ(M)]>0.1, where c₀(M) is the concentration of metal M in the solution PL before mixing, and cₑ(M) is the equilibrium concentration of metal M in the aqueous phase after mixing and reaching the equilibrium,
separating the organic and aqueous phases, and
recovering the metal M from the separated organic phase by re-extraction with an aqueous phase which is neutral, acidic or alkaline, or by precipitation of the metal M by addition of a chemical compound that forms an insoluble compound with the said metal M.

### EXAMPLES

The following examples are provided to assist one skilled in the art to further understand certain embodiments of the present invention. These examples are intended for illustration purposes should not be construed as limiting the scope of the present invention.

In the examples, all values with the unit “%” [=kg/(100 kg)], are intended to be mass fractions of a component in a mixture, or a solute in solution, stated as ratio of the mass of the component or solute under consideration, to the mass of the mixture or solution, unless otherwise specified. When referring to chromatography, “%” stands for a volume fraction.

### Synthetic Examples

The compounds 1 to 26 as listed supra have been synthesised according to the following procedures:

Flash chromatography was done on silica in each case.

Versatic™ Acid 10 (sold by Momentive of Columbus, Ohio, USA) is a synthetic, highly branched C₁₀ tertiary carboxylic acid that consists of a number of isomers; the structure may be represented as:

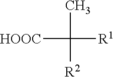

where R¹ and R² are alkyl groups such that the total number of carbon atoms in R¹ and R² is seven. In the structures below that are derived from Versatic® acid 10, the branching is represented as R¹=ethyl and R²=pentyl.

### Pyridine, 2-(5-branched-nonyl-1H-pyrazol-3-yl)-

### (PyrPzVC9) Compound 1

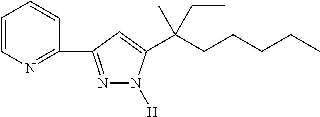 [SMILES: CCCCCC(C)(CC)c1cc(-c2ccccn2)n[nH]1]

To a 1 L, single neck round bottomed flask equipped with a stir bar were charged 69.0 g (250 mmol) of the diketone intermediate (see preparation below), 15.0 g (300 mmol) of hydrazine hydrate, and 350 mL of ethanol. The mixture was heated at reflux for one hour, then was concentrated by rotary evaporation. The residue was diluted with ethyl acetate (700 mL), and washed with water (300 mL), and the organic layer dried with magnesium sulfate. The crude product, 67.4 g, was isolated as a brown oil. Automated flash chromatography (220 g column, from 0% to 50% of a mixture of ethyl acetate (EtOAc) and hexane over 10 minutes, then 50% to 70% over another 10 minutes with product coming off at between eight and thirteen minutes) has been used for purification. Three chromatography iterations were done, with a fourth to separate mixed fractions, giving 43.25 g (0.159 mol, 63.6%) of product as a brown oil. ¹H NMR (400 MHz, CDCl₃) 11.0 (br s, 1H, N—H), 8.6 (d, 1H, ArH), 7.85 (dd, 1H, ArH), 7.70 (t, 1H, ArH), 7.18 (m, 1H, ArH), 6.65 (m, 1H, ═CH—), 1.80-0.70 (m, 19H, aliphatics).

### Pyridine, 2-(3-oxo-branched-dodecanoyl)-

### PyrPzVC9 Diketone Intermediate

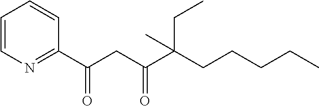 [SMILES: CCCCCC(C)(CC)C(=O)CC(=O)c1ccccn1]

To a 2 L four neck round bottomed flask equipped with a mechanical stirrer, an addition funnel, a condenser, a thermocouple and a nitrogen inlet/outlet, were charged 13.27 g (552.8 mmol) of NaH in 200 mL of toluene. The mixture was brought to 30° C. and 31.84 g (263.2 mmol) of 2-acetylpyridine in 135 mL of toluene was added dropwise over 50 minutes. The mixture was then heated to 40° C. for 1.5 hours, then cooled to 30° C. A solution of 50 g (263.2 mmol) of Versatic® acid chloride in 50 mL of toluene, was then added over 2 hours, during which time the oil bath temperature was maintained at 31° C. The mixture was allowed to stir at room temperature overnight, then the oil bath temperature was increased to 65° C. over 2 hours. An exotherm from 60° C. to 68° C. was observed. After heating at a 65° C. bath temperature for an additional 0.5 hour, TLC showed the reaction to be complete. The mixture was cooled in an ice bath and a solution of 37 g (0.62 mol) of acetic acid in 95 mL of water was added dropwise over 2 hours. After dilution with 300 mL of toluene and 300 mL of water, the organic layer was separated and washed with 200 mL of water. The combined aqueous extracts were backwashed with 300 mL of ethyl acetate and the combined organic layers dried (MgSO₄), filtered and rotary evaporated, giving 73.0 g (263 mmol, 100%) of crude product as a dark oil. ¹H NMR (400 MHz, CDCl₃) 16.2 (m, 1H, enol —O—H), 8.7 (d, 1H, ArH), 8.1 (dd, 1H, ArH), 7.82 (t, 1H, ArH), 7.4 (t, 1H, ArH), 6.95 (m, 1H, enol ═CH—), 1.85-0.70 (m, 19H, aliphatics).

### Branched-Decanoyl Chloride

### Versatic Acid Chloride Intermediate

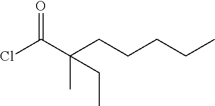

An oven dried 2 L three neck round-bottomed flask was equipped with a thermocouple port, a stir bar and a condenser fitted with a nitrogen inlet/outlet. The gas outlet was connected to a sodium hydroxide-containing scrubber (aqueous solution with a mass fraction of NaOH of 12%). Versatic acid chloride was prepared by reacting Versatic® acid (69.2 g, 0.4 mol) with thionyl chloride (71.4 g, 0.6 mol) in CH₂Cl₂ (69 g) at room temperature overnight. The reaction was ˜85% complete so more thionyl chloride was added (35.7 g, 0.75 mol/mol). The reaction was then refluxed for two hours and the reaction was 94% complete by GC. Another portion of thionyl chloride (17.9 g, 0.38 mol/mol) was added and the reaction was refluxed for 3.5 h. The reaction was judged to be complete by GC at that time. The reaction mixture was concentrated on the rotary evaporator then stirred under high vacuum to yield 71.9 g of material (93%). IR (neat) 1789 cm⁻¹ (C═O). No acid C═O band was observed at 1695 cm⁻¹.

### Pyridine, 2-(5-nonyl-1H-pyrazol-3-yl)-

### (PyrPzC9n) Compound 2

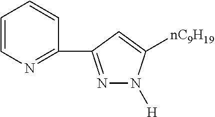

The PyrPzC9n diketone intermediate (see preparation below) (54.4 g, 0.197 mol), was stirred in ethanol (300 ml) in a three neck 1 L round-bottomed flask at 0° C. and a solution of hydrazine hydrate (9.1 g, 0.197 mol) in 100 mL of ethanol was added dropwise over thirty minutes. A white precipitate formed and the mixture was allowed to warm to room temperature, after which time the flask was immersed in an oil bath and heated to 80° C. for one hour. The ethanol was removed by rotary evaporation to give a yellow oil which was dissolved in chloroform (100 ml), washed with water, then dried over Na₂SO₄. The solution was filtered and the chloroform was removed under reduced pressure to give a yellow solid (50 g, 0.184 mol, 93%). ¹H NMR (400 MHz, CDCl₃) 8.61 (m, 1H, Ar—H), 7.79 (d, 1H, Ar—H), 7.68 (t, 1H, Ar—H), 7.17 (m, 1H, ArH), 6.63 (s, 1H, —CH═), 2.68 (t, 2H, —CH₂—), 1.70 (m, 2H, —CH₂—), 1.4-1.2 (m, 12H, (—CH₂-)6), 0.90 (t, 3H, —CH₃).

### Pyridine, 2-(3-oxononanoyl)-

### PyrPzC9n Diketone Intermediate

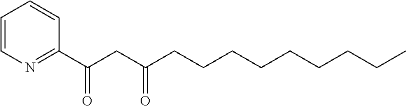 [SMILES: CCCCCCCCCC(=O)CC(=O)c1ccccn1]

Under nitrogen atmosphere, sodium methoxide (13.2 g, 244 mmol) was dispersed in dry THF (300 mL) in a three neck 1 L round-bottomed flask. Undecanone (37.23 g, 219 mmol) was dissolved in THF (100 mL) and added to the slurry dropwise, followed by methyl picolinate (30 g, 219 mmol) in THF (100 mL). The reaction was heated at reflux for twenty-six hours, and then cooled to room temperature. Acetic acid (15 g, 250 mmol) was dissolved in THF (100 mL) and added dropwise over 10 min and the reaction stirred for 20 min at room temperature. THF was removed by rotary evaporation and the residue was dissolved in ethyl acetate (100 mL) and water (100 mL). The organic phase was separated from the aqueous phase and washed with water (2×25 mL), and then dried over Na₂SO₄. The solution was filtered and solvent removed under reduced pressure to give a yellow oil, 56.4 g. ¹H NMR (400 MHz, CDCl₃) 15.7 (br s, 1H, enol —O—H), 8.61 (m, 1H, Ar—H), 8.02 (d, 1H, Ar—H), 7.78 (t, 1H, Ar—H), 7.32 (m, 1H, ArH), 6.78 (s, 1H, —HC═), 2.40 (t, 2H, —CH₂—), 1.65 (m, 2H, —CH₂—), 1.4-1.15 (m, 12H, (—CH₂-)6), 0.82 (t, 3H, —CH₃).

### Pyridine, 2-1H-pyrazol-1-yl

### (Pyr-N-PzH) Compound 3

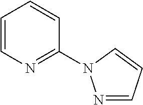 [SMILES: c1ccc(-n2cccn2)nc1]

To a 250 mL single-neck round bottomed flask were charged 15.0 g (95 mmol) of 2-bromopyridine, 25.0 g (367 mmol, 3.86 eq.) of pyrazole, and 45 mL of xylenes. The mixture was heated at reflux for eight hours, then was cooled to room temperature. The resulting mixture was dissolved in dichloromethane, and the organic layer washed four times with 250 mL of water (until no pyrazole was observed by GC). Drying over magnesium sulfate, filtering and rotary evaporation gave 12.0 g (82.6 mmol, 87%) of the product as a white solid. ¹H NMR (400 MHz, CDCl₃) 8.59 (m, 1H, Pyridyl 6-H), 8.39 (s, 1H, Pyrazole 5-H), 7.90 (d, 1H, Pyridyl 3-H), 7.80 (m, 1H, Pyridyl 4-H), 7.73 (s, 1H, Pyrazole 3-H), 7.15 (s, 1H, Pyridyl 5-H), 6.45 (m, 1H, Pyrazole 4-H).

### Pyridine, 2,6-bis(5-nonyl-1H-pyrazol-3-yl)-

### Pyrbis(PzC₉n) Compound 4 (Compound Reported in Zhou and Pesic 1997)

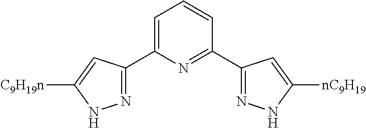

A 250 mL three neck round bottomed flask was equipped with a mechanical stirrer and condenser with attached N2 inlet/outlet. The tetraketone intermediate described below (13.12 g, 0.28 mol) was diluted with methanol (46 mL). Hydrazine monohydrate (2.94 g, 0.59 mol) was then added. An exotherm was observed just after mixing. The pot temperature climbed from 21° C. to 38° C. The reaction was then placed in an 80° C. oil bath oil bath for 2.5 h. Additional hydrazine hydrate (0.882 g, 0.018 mol) was added and the reaction was refluxed for two more hours. The reaction was allowed to cool to room temperature then concentrated on the rotary evaporator. The residue was partitioned between dichloromethane and water. The organic layer was then washed with brine and dried with molecular sieves. The material was then concentrated on the rotary evaporator then placed under high vacuum to yield 12.84 g (98%) of product. ¹H NMR (400 MHz, CDCl₃) δ 12.4 (br s, 2H, N—H) 7.6 (t, 1H, ArH), 7.4 (br s, 2H, ArH), 6.5 (br s, 2H, pyrazole-H), 2.7 (t, 4H, 2CH₂—C═), 1.7 (t, 4H, 2CH₂CH₂—C═), 1.3 (m, 24H, aliph), 0.8 (t, 6H, Me)

### Pyridine, 2,6-bis(3-oxononanoyl)-

### Pyrbis(PzC₉n) Tetraketone Intermediate (Compound Reported in Zhou and Pesic 1997)

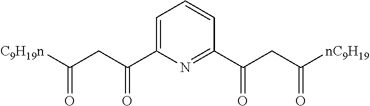

A 1 L four neck round bottomed flask was equipped with two dropping funnels, a thermocouple, and a nitrogen inlet/outlet. Sodium methoxide (12.04 g, 0.223 mol) was transferred to the flask. Anhydrous THF (165 mL) was added through the dropping funnel. 2,6-Dimethoxypyridine (19.78 g, 0.101 mol) was suspended in THF (100 mL). The undecanone (34.39 g, 0.202 mol) was dissolved in THF (65 mL). The 2,6-dimethoxypyridine and undecanone were added dropwise at the same time over 20 min. The reaction mixture was heated with an internal temperature around 63° C. for three days. The reaction was cooled to room temperature then sodium methoxide (1.2 g, 0.022 mol) was added followed by undecanone (3.4 g, 0.02 mol). The reaction was then heated two more days. The reaction was carefully quenched by the slow dropwise addition of 25% acetic acid in water (60 mL). Single drops were added at first and vigorous bubbling was seen. The reaction was concentrated to a thick slurry. The material was partitioned between dichloromethane and water. The material was washed with brine, dried with Na₂SO₄, then concentrated under reduced pressure. The product was recrystallized from hexanes to yield 18.24 g (38%) of product. ¹H NMR (400 MHz, CDCl₃) δ 12.4 (br s, 2H, N—H) 7.6 (t, 1H, ArH), 7.4 (d, 2H, ArH), 6.5 (br s, 2H, pyrazole-H), 2.7 (t, 4H, 2CH₂—C═), 1.7 (t, 4H, 2CH₂CH₂—C═), 1.3 (m, 24H, aliph), 0.8 (t, 6H, Me)

### Pyrazine, 2-(5-branched-nonyl-1H-pyrazol-3-yl)-

### (PyzPzVC9) Compound 5

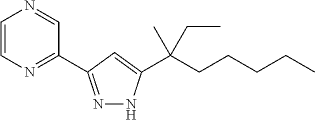 [SMILES: CCCCCC(C)(CC)c1cc(-c2cnccn2)n[nH]1]

A 100 mL single neck reaction vessel was equipped with a stir bar, a condenser, and a nitrogen inlet/outlet. The diketone (preparative method described below) (4.34 g, 0.16 mol) was diluted with ethanol (30 mL) and transferred to the reactor. Hydrazine monohydrate (0.86 g 0.16 mol) was then added. The reaction flask was then placed in a preheated 80° C. oil bath for one hour. The reaction was allowed to cool to room temperature, then was concentrated on a rotary evaporator. The residue was partitioned between hexanes and water. The organic layer was then washed with brine and dried with sodium sulfate. The material was then filtered and concentrated on a rotary evaporator to yield 3.79 g (89%) of crude pyrazine-pyrazole after high vacuum treatment. ¹H NMR (400 MHz, CDCl₃) δ 10.5 (br s, 1H, N—H), 9.3 (m, 1H, ArH), 8.6 (s, 1H, ArH), 8.4 (s, 1H, ArH), 6.7 (m, 1H, pyrazole-H), 2.0-0.6 (m, 19H, aliphatic).

### Pyrazine, 2-(3-oxo-branched-dodecanoyl)-

### PyzPzVC9 Diketone Intermediate

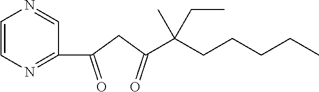 [SMILES: CCCCCC(C)(CC)C(=O)CC(=O)c1cnccn1]

A 2 L three neck round bottomed flask was equipped with a condenser, a mechanical stirrer, a thermocouple and a nitrogen inlet/outlet. A 1000 mL spacer was added between the flask and the condenser to give room for frothing. Solid 95% sodium hydride (8.25 g, 0.344 mol) was weighed into the flask and diluted with dry toluene (120 mL). The pot was warmed to 35° C. 2-Acetyl pyrazine (20 g, 0.164 mol) was added by solid addition funnel over 1 hour and 25 minutes while maintaining the reaction temperature below 40° C. The reaction was stirred at 40° C. for an hour. Versatic® acid chloride (preparative method described above) was diluted with 23 mL of toluene then added dropwise while maintaining the reaction temperature at 20° C. over the course of forty minutes. The reaction was then refluxed for sixty hours, and then cooled. The mixture was quenched by the dropwise addition of a solution containing 33 mL of glacial acetic acid in 98 mL of water over a 2.5 h period. The aqueous layer was then removed and the organic layer washed with brine. The material was concentrated under reduced pressure to yield 50 g of crude material which was subjected to automated flash chromatography (220 g column, gradient: from 0% to 15% ethyl acetate/hexanes over thirty minutes with a flow of 150 mL/min). The column yielded 4.34 g (9.5%) of product. ¹H NMR (400 MHz, CDCl₃) δ 9.3(br s, 1H, ArH), 8.7 (br s, 1H, ArH), 8.6 (br s, 1H, ArH), 2.1-0.7 (m, 19H, aliph).

### Pyridine, 2-(5-branched-nonyl-1,2-oxazole-3-yl)-

### (Pyr-Isox-VC9-A) Compound 6

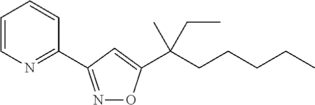 [SMILES: CCCCCC(C)(CC)c1cc(-c2ccccn2)no1]

To a 1 L, three neck round bottomed flask equipped with a stir bar were charged 55.70 g (202.5 mmol) of the PyrPzVC9 diketone intermediate (see preparation above), 14.1 g (202.5 mmol) of hydroxylamine hydrochloride, and 160 mL of methanol. The mixture was heated to reflux for one hour, after which time TLC showed complete conversion of the diketone to two major products. The mixture was concentrated by rotary evaporation, diluted with ethyl acetate (300 mL) and washed with water (300 mL). After drying (magnesium sulfate), filtration and rotary evaporation, the residue (ca. 55 g) was purified using automated flash chromatography (220 g column, 0% ethyl acetate/Hexane until six minutes, from 0% to 10% ethyl acetate/Hexane over 9 minutes, hold at 10% for 11 min, then used a gradient from 10% to 50% over 1.5 min, and hold at 50% for 7.5 min). Two column iterations were run, with a third run for purification of mixed fractions. Compound A comes off at ca. from 13 min to 16 min, giving 11.75 g (43 mmol, 21%) of a yellow oil. ¹H NMR (400 MHz, CDCl₃) 8.69 (d, 1H, Pyridyl 6-H), 8.09 (d, 1H, Pyrazole 5-H), 7.78 (t, 1H, Pyridyl 3-H), 7.32 (m, 1H, Pyridyl 4-H), 6.63 (m, 1H, ═CH—), 1.90-0.80 (m, 19H, aliphatics).

### Pyridine, 2-(3-branched-nonyl-1,2-oxazole-5-yl)-

### (Pyr-Isox-VC9-B) Compound 7

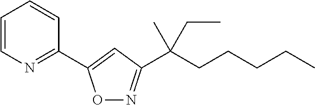 [SMILES: CCCCCC(C)(CC)c1cc(-c2ccccn2)on1]

Compound B was isolated from the above mixture. Compound B came off at from 17 min to 25 min, giving 5.73 g (21 mmol, 10%) of a yellow oil. ¹H NMR (400 MHz, CDCl₃) 8.68 (d, 1H, Pyridyl 6-H), 7.90 (d, 1H, Pyrazole 5-H), 7.82 (t, 1H, Pyridyl 3-H), 7.32 (m, 1H, Pyridyl 4-H), 6.83 (m, 1H, ═CH—), 1.80-0.70 (m, 19H, aliphatics).

### Pyridine, 2-(5-(1-butylheptyl)-1,2,4-oxadiazol-3-yl)-

### (Pyr124OdC11) Compound 8

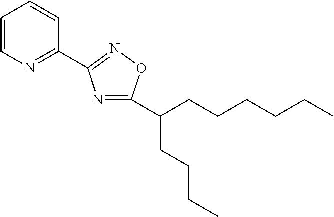 [SMILES: CCCCCCC(CCCC)c1nc(-c2ccccn2)no1]

A 500 mL three neck round bottomed flask was equipped with a mechanical stirrer, thermocouple and condenser with attached N2 inlet/outlet. Methyl 2-butyloctanoate (50 g, 0.234 mol), xylene (40 mL), the amidoxime intermediate (preparative method described below) (32.2 g, 0.234 mol) and potassium carbonate (38.9 g, 0.28 mol) were transferred to the reaction vessel. The mixture was heated in a 148° C. oil bath for 24 h, and then was cooled to room temperature. The mixture was cooled to room temperature then partitioned between hexanes and water. The aqueous layer was extracted with hexanes. The combined organic layers were washed with a 7% strength aqueously diluted H₂SO₄, water, then brine. After drying with sodium sulfate, the organic layer was filtered and concentrated under reduced pressure. The material was purified by automated flash chromatography in three portions; eluent in each case was a gradient mixture of from 0% to 15% ethyl acetate/hexanes. The first column was 40 g, gradient time: sixteen minutes, flow 40 mL/min. There were 2×220 g columns, each with gradient time thirty minutes and flow 150 mL/min. Yield of the combined product fractions 41.16 g (58%) of product. ¹H NMR (400 MHz, CDCl₃) δ 8.8 (dd, 1H, ArH), 8.1 (dd, 1H, ArH), 7.8 (t, 1H, ArH), 7.3 (m, 1H, ArH), 3.1 (m, 1H, CH—C═), 1.8 (m, 2H, CH₂CH—C═), 1.7 (m, 2H, CH₂CH—C═), 1.4-1.1 (m, 12H, aliph), 0.8 (m, 6H, Me).

### Methyl 2-Butyloctanoate

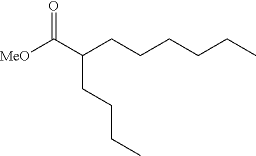

2-Butyl octanoic acid (50 g, 0.25 mol) was charged to a one neck, 250 mL round bottomed flask equipped with stir bar and condenser supporting a nitrogen inlet/outlet. Methanol (100 g, 3.1 mol) was added, followed by a catalytic amount (2 mL) of acetyl chloride. The solution was refluxed for ten hours under N2. The ester was then concentrated under reduced pressure to yield 53.54 g (100%) of the product. ¹H NMR (400 MHz, CDCl₃) δ 3.6 (s, 3H, OMe), 2.3 (m, 1H, CHC═O), 1.6 (m, 1H, CH₂CH), 1.4 (m, 1H, CH₂CH), 1.2 (m, 12H, aliph), 0.85 (m, 6H, Me).

### Pyridine, 2-(5-(1-butylheptyl)-1,2,4-oxadiazol-3-yl)-

### (Pyr124OdC8) Compound 9

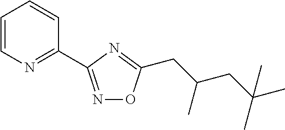 [SMILES: CC(Cc1nc(-c2ccccn2)no1)CC(C)(C)C]

A 500 mL three neck round bottomed flask was equipped with a mechanical stirrer, thermocouple and condenser with attached N2 inlet/outlet. The methyl 3,5,5-trimethyl hexanoate (preparative method described below) (40 g, 0.21 mol), toluene (200 mL), amidoxime (preparative method described in Section 0) (28.9 g, 0.21 mol) and potassium carbonate (34.8 g, 0.25 mol) were transferred to the reaction vessel. The reaction was heated to reflux at ca. 93° C. overnight. The reaction was partitioned between toluene and water. The aqueous layer was extracted with toluene. The combined organics were washed with 7% strength aqueously diluted H₂SO₄, and then dried with sodium sulfate, filtered and concentrated under reduced pressure to yield 38.45 g (71%) of product. ¹H NMR (400 MHz, CDCl₃) δ 8.8 (dd, 1H, ArH), 8.1 (dd, 1H, ArH), 7.8 (t, 1H, ArH), 7.4 (m, 1H, ArH), 3.0 (m, 1H, CH₂—CH═), 2.8 (m, 1H, CH₂—CH═), 2.3 (m, 1H, (Me)C—H), 1.4 (m, 1H, CH₂C(Me)₃), 1.2 (m, 1H, CH₂C(Me)₃), 1.1 (d, 3H, MeC—H), 0.9 (s, 9H, C(Me)₃).

### Methyl 3,5,5-Trimethylhexanoate

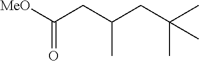

3,5,5-trimethylhexanoic acid (50 g, 0.26 mol) was transferred into a one neck 250 mL round bottomed flask equipped with stir bar and a condenser supporting a nitrogen inlet/outlet. Methanol (78 g, 2.4 mol) was added followed by a catalytic amount (0.76 g) of acetyl chloride. The solution was refluxed for four hours under N2. The ester was then concentrated under reduced pressure to yield 43.29 g (80%) of material. ¹H NMR (400 MHz, CDCl₃) δ 3.7 (s, 3H, Me), 2.3 (m, 1H, CH₂—C═O), 2.1 (m, 1H, CH₂—C═O), 1.3 (m, 2H, CH₂-tBu, and CH(CH₂)Me), 1.1 (m, 1H, CH₂-tBu), 1.0 (d, 3H, Me), 0.9 (s, 9H, tBu).

### Pyridine, 2-(5-branched-nonyl-1,2,4-oxadiazole-3-yl)-

### (Pyr124OdVC9) Compound 10

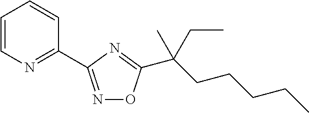 [SMILES: CCCCCC(C)(CC)c1nc(-c2ccccn2)no1]

Method 1: a 500 mL four neck flask was equipped with a mechanical stirrer, a condenser, a thermocouple and a nitrogen inlet outlet, and charged with 20.0 g (67.34 mmol) of the amidoxime ester intermediate (preparative method described below), 40 mL of ethylene glycol, and 16.7 g of 0.4 nm (4 Å) molecular sieves. The mixture was heated with gentle stirring for one hour. The TLC showed a slightly less polar product spot and more polar impurity spot. The mixture was then filtered and the sieves washed with ethyl acetate. The solution was concentrated, diluted with dichloromethane (300 mL) and washed with water (200 mL). The organic layer was then dried with sodium sulfate, filtered and rotary evaporated, giving an oil. This crude product was purified by automated flash chromatography in two portions, using a 120 g column, and the following gradient: from 0% to 25% of ethyl acetate in a mixture of ethyl acetate/hexane over one minute, holding at 25% ethyl acetate for 8 minutes, then a gradient of from 25% to 50% of ethyl acetate over one minute, with impure product eluting at from 3 min to 5 min. A final column was run to repurify the impure product, giving 2.01 g (7.2 mmol, 10.7 g) of the oxadiazole as a yellow oil. ¹H NMR (400 MHz, CDCl₃) 8.83 (d, 1H, Ar—H), 8.15 (d, 1H, Ar—H), 7.85 (t, 1H, Ar—H), 7.40 (m, 1H, Ar—H), 1.90-0.80 (m, 19H, aliphatics).

### Branched-Nonanoic Acid, (Imino-2-Pyridinylmethyl)Azanyl Ester Intermediate

### Amidoxime Branched-Nonanoate Ester Intermediate

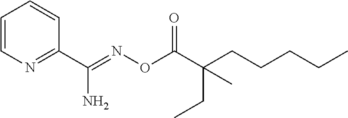 [SMILES: CCCCCC(C)(CC)C(=O)O/N=C(\N)c1ccccn1]

To a 500 mL three-necked round bottom was added 22.0 g (160.6 mmol) of the amidoxime intermediate (see preparation method below) in 120 mL of dichloromethane. Triethylamine (22.4 mL) was added dropwise at 0° C. after which time the mixture was stirred for fifteen minutes. To this mixture were added 30.51 g (160.6 mmol) of Versatic® acid chloride (see preparation method above) in 20 mL of dichloromethane at 0° C. The mixture was allowed to stir overnight at room temperature, after which time TLC showed one major spot. The mixture was diluted with 300 mL of dichloromethane, and washed with 2×200 mL of water, followed by brine (200 mL). The organic layer was dried over sodium sulfate, filtered and rotary evaporated, giving 46.10 g of a brown oil, which solidified on standing. ¹H NMR (400 MHz, CDCl₃) 8.58 (d, 1H, Ar—H), 8.21 (d, 1H, Ar—H), 7.75 (t, 1H, Ar—H), 7.78 (t, 1H, Ar—H), 7.37 (t, 1H, Ar—H), 6.3 (br s, 2H, —NH₂).

### 2-Pyridinecarboximidamide, N-Hydroxy-

### Amidoxime Intermediate

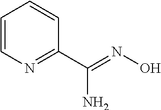

To a 1 L single neck round-bottomed flask equipped with a stir bar were charged 40 g (384 mmol) of 2-pyridinecarbonitrile, 26.7 g (384 mmol) of hydroxylamine hydrochloride, 107 mL (768 mmol) of triethylamine and 300 mL of ethanol. The flask was fitted with a reflux condenser and the mixture heated under reflux for three hours (TLC showed no remaining starting material). After concentration on a rotary evaporator, the residue was slurried in a mixture of 300 mL of dichloromethane and 300 mL of water. The slurry was filtered and the product cake washed several times with water. The organic layer washed with 2×300 mL of water. The aqueous layers were backwashed with 50 mL of dichloromethane and the combined organic layers dried over magnesium sulfate, filtered and rotary evaporated, giving a white solid. The solids were combined and dried in a reduced pressure oven, giving 52.5 g (100%) of product, m.p. 116° C. ¹H NMR (400 MHz, DMSO-d6) 9.9 (s, 1H, —OH), 8.53 (m, 1H, Ar—H), 7.85 (d, 1H, Ar—H), 7.78 (t, 1H, Ar—H), 7.38 (m, 1H, Ar—H), 5.82 (br s, 2H, —NH₂), 1.90-0.85 (m, 19H, aliphatics).

### Pyridine, 2-(5-branched-nonyl-1,2,4-oxadiazole-3-yl)-

### (Pyr124OdVC9) Compound 10

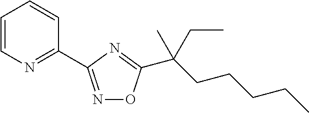 [SMILES: CCCCCC(C)(CC)c1nc(-c2ccccn2)no1]

Method 2: a 100 mL four neck round bottomed flask was equipped with a mechanical stirrer, thermocouple, stopper and condenser with attached N2 inlet/outlet. The methyl ester of Versatic® acid (preparative method described below) (6.7 g, 0.036 mol), xylene (30 mL), amidoxime (4.98 g, 0.036 mol) and potassium carbonate (5.52 g, 0.039 mol) were transferred to the reaction vessel. The reaction mixture was heated in a 150° C. oil bath overnight. The internal temperature was about 136° C. overnight. The reaction was partitioned between hexanes and water. The aqueous layer was extracted with hexanes. The combined organics were washed with water, then brine, and then dried with sodium sulfate, filtered and concentrated under reduced pressure to yield 7.34 g, of mixed starting material and product. The product was not isolated. Other means of synthesizing the desired material were then pursued (see above).

### Versatic Acid Methyl Ester

### Methyl Branched-Decanoate

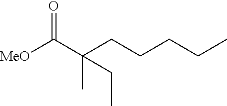

Methanol (30 g, 0.93 mol) was transferred to a four neck 250 mL flask equipped with a mechanical stirrer, thermocouple, dropping funnel and condenser connected to a nitrogen inlet/outlet. Triethyl amine (5.33 g, 0.053 mol) was added to the reaction vessel. Versatic® acid chloride (preparative method described above) (10 g, 0.053 mol) was added to the reaction vessel dropwise. The reaction was shown to be complete by gas chromatography after two hours. The reaction mixture was concentrated under reduced pressure to remove the methanol. The residue was partitioned between hexanes and water. The organic layer was washed with water and brine, then dried with molecular sieves. After filtration, the solvent was then removed under reduced pressure to yield 6.77 g (69%) of product as an oil. ¹H NMR (400 MHz, CDCl₃) δ 3.65 (m, 3H, OCH₃), 2.0-0.7 (m, 19H, aliph).

### Pyridine, 2-(5-(1-(3-methylhexyl)-6-methylnon-1-yl)-1,2,4-oxadiazol-3-yl)-

### (Pyr124OdC17NissN) Compound 11

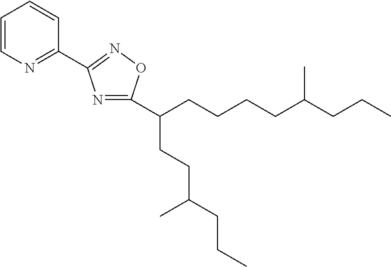 [SMILES: CCCC(C)CCCCC(CCC(C)CCC)c1nc(-c2ccccn2)no1]

A 250 mL four neck round bottomed flask was equipped with a mechanical stirrer, thermocouple, stopper and condenser with attached N2 inlet/outlet. Methyl isostearate (preparative method described below) (16.77 g, 0.056 mol), toluene (50 mL), amidoxime (preparative method described above) (7.68 g, 0.056 mol) and potassium carbonate (9.27 g, 0.067 mol) were transferred to the reaction vessel. The reaction mixture was heated in a 125° C. oil bath for 19.5 hours. The reaction mixture still comprised a significant amount of starting material as shown by gas chromatography. The reaction mixture was concentrated under reduced pressure to remove toluene. Xylene (50 mL) was added. The reaction mixture was then heated in a 155° C. oil bath for twenty-seven hours. The reaction mixture was partitioned between hexanes and water. The aqueous layer was extracted with hexanes. The combined organics were washed with water, then brine, and then dried with sodium sulfate, filtered and concentrated under reduced pressure. The material was purified by automated flash chromatography (220 g column, elution over 30 minutes with a gradient of from 0% to 10% ethyl acetate/hexanes and a flow of 150 mL/min) to yield 9.45 g, (44%) of product. ¹H NMR (400 MHz, CDCl₃) δ 8.8 (dd, 1H, ArH), 8.1 (dd, 1H, ArH), 7.8 (t, 1H, ArH), 7.4 (m, 1H, ArH), 3.3-3.0 (m, 1H, CH—C═), 2.0-1.6 (m, 4H, CH₂—CH═), 1.5-1.0 (m, 16H, aliph), 0.9-0.6 (m, 14H, aliph).

### Methyl Ester of Isostearic N Acid

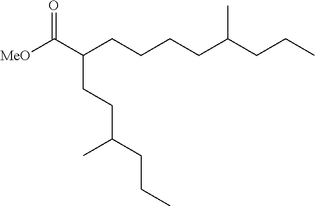 [SMILES: CCCC(C)CCCCC(CCC(C)CCC)C(=O)OC]

Isostearic N acid (branched C18 alkanoic acid mixture, 2-[3-methylhexyl]-7-methyldecanoic acid as main isomer, 60 g, 0.21 mol, available from Nissan Chemical Company) was charged to a 2 neck 250 mL round bottomed flask equipped with stir bar, stopper, nitrogen inlet/outlet attached to a riser. A scrubber was attached to the N2 outlet to remove HCl fumes. To the isostearic acid was added thionyl chloride (37.6 g, 0.32 mol) in CH₂Cl₂ (60 g) at room temperature. The mixture was stirred overnight and rotary evaporated, giving 63.25 g (99%) of isostearyl chloride after high vacuum treatment. Methanol (40 g, 1.25 mol) was transferred to a four neck 150 mL flask equipped with a mechanical stirrer, thermocouple, dropping funnel and condenser connected to a nitrogen inlet/outlet. Triethylamine (8 g, 0.079 mol) was added to the reaction vessel. The solution was cooled to 3.7° C. using an ice bath. A portion of the isostearic acid chloride above (20.21 g, 0.066 mol) was added dropwise over ten minutes. The reaction mixture was warmed to room temperature and stirred overnight. The reaction was shown to be complete by gas chromatography. The reaction mixture was concentrated under reduced pressure to remove the methanol. The residue was partitioned between hexanes and water. The organic layer was washed with water, brine, then dried with molecular sieves. After filtration, the solvent was then removed under reduced pressure to yield 16.8 g (85%) of the product as a liquid. ¹H NMR (400 MHz, CDCl₃) δ 3.65 (s, 3H, OMe), 2.5-2.3 (m, 1H, CHC═O), 1.7-0.7 (m, 35H, aliph).

### Pyridine, 2-(5-(heptadeca-8,11-diene-1-yl))-1,2,4-oxadiazole-3-yl)-

### (Pyr124OdC17soy) Compound 12

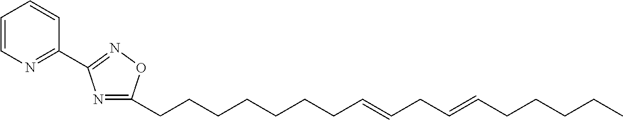 [SMILES: CCCCC/C=C/C/C=C/CCCCCCCc1nc(-c2ccccn2)no1]

A 250 mL four neck round bottomed flask was equipped with a mechanical stirrer, stopper and condenser with attached N2 inlet/outlet and thermocouple. Proctor & Gamble SE1885 soybean methyl ester (30 g, 0.1 mol), xylene (100 mL), amidoxime (13.7 g, 0.1 mol) and potassium carbonate (16.7 g, 0.12 mol) were transferred to the reaction vessel. The mixture was heated in a 125° C. oil bath for 24.5 h. The internal temperature of the reaction was maintained around 108° C. Water was added to the reaction vessel. The material became a mud-like suspension. The aqueous layer was extracted with hexanes. The combined organics were washed with water then brine, dried with sodium sulfate, filtered and concentrated under reduced pressure to yield 33.95 g (85 mmol, 85%) of product, which was shown to be about 88% pure by GC. The major contaminant was the ester starting material. The material was used without further purification. ¹H NMR (400 MHz, CDCl₃) δ 8.8 (dd, 1H, ArH), 8.1 (dd, 1H, ArH), 7.8 (t, 1H, ArH), 7.4 (m, 1H, ArH), 5.3 (m, 4H, CH₂—CH═), 3.0 (t, 2H, aliph), 2.8-2.6 (m, 2H, aliph), 2.3 (m, 2H, aliph), 2.0 (m, 4H, aliph), 1.9 (m, 2H, aliph), 1.4-1.2 (m, 20H, aliph), 0.8 (s, 3H, Me).

### Pyridine, 2-(5-branched-nonyl-1,3,4-oxadiazole-2-yl)-

### (Pyr134OdVC9) Compound 13

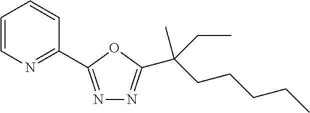 [SMILES: CCCCCC(C)(CC)c1nnc(-c2ccccn2)o1]

To a 2 L three neck round-bottomed flask equipped with a mechanical stirrer and an addition funnel were charged 29.0 g (211.7 mmol) of the hydrazide intermediate (preparative method given below) and 290 mL of dichloromethane. The mixture was cooled in an ice bath and 88.5 mL of triethylamine (64.25 g, 635 mmol, 3 eq.) was added dropwise. A precipitate formed, and the mixture was stirred for fifteen minutes. Versatic® acid chloride (40.2 g, 211.7 mmol, 1 eq.) (preparative method described above), in 70 mL of dichloromethane was added dropwise at 0° C. The mixture was then stirred for two hours at room temperature, then cooled again to 0° C. in an ice bath. Tosyl chloride (40.36 g, 211.7 mmol, 1 mol/mol) was added via solid addition funnel over thirty minutes, keeping the temperature between 6° C. and 10° C. The mixture was then stirred at room temperature overnight. The next day, 20% of the initial charges of triethylamine and tosyl chloride were added to the mixture and the mixture stirred for three hours to consume remaining starting material. The mixture was diluted with 300 mL of dichloromethane and washed with 300 mL of water. The organic layer was dried over sodium sulfate, filtered and rotary evaporated, giving a residue that was purified by automated flash chromatography in three portions, using a 220 g column, and the following gradient: 0-0% ethyl acetate/hexane for eight minutes, going to 30% ethyl acetate over two minutes, holding at 30% ethyl acetate for five minutes, going to 50% ethyl acetate over five minutes and holding at 50% ethyl acetate until twenty-six minutes. Product elutes at from 11 min to 20 min. A 56.24 g (206 mmol, 97%) portion of product as a brown oil was isolated. ¹H NMR (400 MHz, CDCl₃) 8.81 (m, 1H, Ar—H), 8.24 (d, 1H, Ar—H), 7.88 (t, 1H, Ar—H), 7.45 (m, 1H, Ar—H), 2.10-0.70 (m, 19H, aliphatics).

### 2-Pyridinecarboxylic Acid Hydrazide

### Pyridine 2-Hydrazide Intermediate

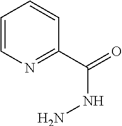

To a 500 mL three neck round-bottomed flask equipped with a mechanical stirrer, a reflux condenser, and a nitrogen inlet/outlet was added methyl picolinate (40 g, 0.292 mol), hydrazine hydrate (17.5 g, 0.35 mol) and ethanol (120 mL). The resulting mixture was heated under reflux for 1 h. The solvent was removed under reduced pressure and the resulting residue dried to give the product 40 g (0.292 mol, 100%) as a white solid. ¹H NMR (400 MHz, CDCl₃) 8.99 (br s, 1H, —N—H), 8.51 (m, 1H, Ar—H), 8.13 (m, 1H, Ar—H), 7.82 (m, 1H, Ar—H), 7.40 (m, 1H, Ar—H) 4.08 (br s, 2H, —NH₂).

### Pyridine, 2-(5-(1-hexylnonyl)-1,3,4-oxadiazole-2-yl)-

### Pyr134OdC15 Compound 14

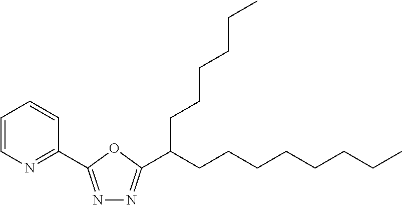 [SMILES: CCCCCCCCC(CCCCCC)c1nnc(-c2ccccn2)o1]

A 500 mL four neck round bottomed flask was equipped with a mechanical stirrer, thermocouple, dropping funnel and condenser with attached N2 inlet/outlet. 2-Pyridinecarboxylic acid hydrazide (preparative method described above) (20 g, 0.146 mol) was suspended in dichloromethane (200 mL) the slurry was cooled with an ice bath to 2.8° C. Triethylamine (44.3 g, 0.438 mol) was added dropwise over fifteen minutes. 2-Hexyldecanoyl chloride (40.37 g, 0.146 mol; preparation described below) was diluted with 45 mL of dichloromethane then added dropwise over thirty minutes at from 3.8° C. to 9.5° C. The pot was then allowed to warm to room temperature. Within two hours the hydrazide was completely consumed. The reaction was cooled with an ice bath. Tosyl chloride (27.8 g, 0.146 mol) was added over twenty-two minutes using a solid addition funnel. The reaction was stirred at room temperature over the weekend. A small amount of uncydized material remained. Tosyl chloride (2.8 g, 0.0146 mol) in dichloromethane was added. The reaction was stirred for four more hours but did not go to completion. The mixture was then partitioned between dichloromethane and water. The organic layer was washed as follows: twice with sodium bicarbonate solution, then brine, then water. The material was then purified by automated flash chromatography (220 g column with a gradient of from 0% to 20% ethyl acetate/hexanes over fifteen minutes with a flow of 200 mL/min) to yield a total of 26.9 g (24%). ¹H NMR (400 MHz, CDCl₃) b 8.8 (dd, 1H, ArH), 8.3 (dd, 1H, ArH), 7.9 (t, 1H, ArH), 7.4 (m, 1H, ArH), 3.1 (m, 1H, C—H), 1.9 (m, 2H, CH₂—C═), 1.7 (m, 2H, CH₂—C═), 1.3 (m, 20H, aliph), 0.85 (m, 6H, Me).

### 2-Hexyldecanoyl Chloride

### (Isopalmitoyl Chloride) (Intermediate)

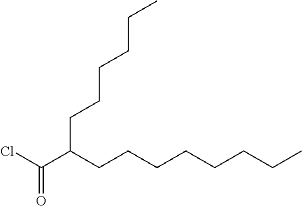 [SMILES: CCCCCCCCC(CCCCCC)C(=O)Cl]

Isopalmitoyl chloride was prepared by stirring isopalmitic acid (isomer mixture comprising 2-hexyldecanoic acid and other C16 isomers, 60 g, 0.232 mol, Nissan Chemical Company) with thionyl chloride (41 g, 0.35 mol) in CH₂Cl₂ (60 g) at room temperature overnight in a 2 neck 250 mL round bottomed flask equipped with stir bar and N2 inlet/outlet attached to a riser and stopper. A scrubber was attached to the N2 outlet to remove HCl fumes. The reaction yielded 62.5 g of acid chloride (quantitative). ¹H NMR (400 MHz, CDCl₃) 2.8 (m, 1H, CH(CH₂R₁)(CH₂R₂), 1.8 (m, 2H, CH₂C), 1.6 (m, 2H, CH₂C), 1.3 (m, 20H, aliph), 0.9 (m, 6H, Me).

### Pyridine, 2-(5-branched-nonyl-1,2,4-triazole-3-yl)-

### (Pyr124TzVC9) Compound 15

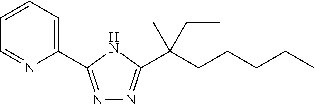 [SMILES: CCCCCC(C)(CC)c1nnc(-c2ccccn2)[nH]1]

To a 1 L four neck round-bottomed flask equipped with a stir bar and reflux condenser were charged 84.0 g (290 mmol) of the branched-nonanoyl amidrazone intermediate (preparative method described below), and 330 mL of ethylene glycol. The mixture was heated at 180° C. for two hours. TLC showed some remaining starting material, so the mixture was heated at 195° C. for two additional hours. The solvent was removed by distillation, dissolved in ethyl acetate then washed with water, concentrated and purified in four portions by automated flash chromatography (220 g column, gradient of from 0% to 50% ethyl acetate/hexane over ten minutes, hold at 50% for 7.5 min, a gradient of from 50% to 90% over 2.5 min, hold at 90% for five minutes; product elutes at from 8 min to 15 min) to give the product, 44.9 g (165 mmol, 57%) as a white solid, having a melting temperature of 70° C. A considerable amount of less polar byproduct was also isolated and analysis showed the material to be pyridyl-1,3,4-oxadiazole VC9 by LC/MS and ¹H-NMR. ¹H NMR (400 MHz, CDCl₃) 12.2 (br s, 1H, N—H), 8.73 (d, 1H, Ar—H), 8.24 (d, 1H, Ar—H), 7.82 (t, 1H, Ar—H), 7.37 (m, 1H, Ar—H), 2.05-0.70 (m, 19H, aliphatics).

### 2-Pyridinecarboximidic Acid, N-Branched-Nonanoyl, Hydrazide

### N-Branched-Nonanoyl Amidrazone Intermediate

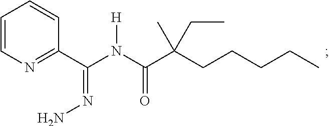 [SMILES: CCCCCC(C)(CC)C(=O)N/C(=N/N)c1ccccn1]

To a 2 L three neck round-bottomed flask equipped with a mechanical stirrer, a reflux condenser, an addition funnel and a nitrogen inlet/outlet were charged the amidrazone intermediate (preparative method described below) (40 g, 294 mmol), sodium carbonate (31.2 g, 294 mmol), dimethylacetamide (250 mL) and tetrahydrofuran (80 mL). The mixture was cooled to 0° C. and Versatic® acid chloride (preparative method described above) (55.9 g; 294 mmol) in dimethylacetamide (85 mL) was added slowly. The mixture was stirred at room temperature for three hours. The thick mixture was concentrated, and water was added (200 mL). The mixture was filtered, washed with water and dried to give the product, 85 g (294 mmol, 100%) as a solid. ¹H NMR (400 MHz, CDCl₃) 8.50 (m, 1H, Ar—H), 8.28 (d, 1H, Ar—H), 8.25 (br s, 1H, —N—H), 7.70 (m, 1H, Ar—H), 7.30 (m, 1H, Ar—H), 6.08 (br s, H, —NH), 1.90-0.80 (m, 19H, aliphatics).

### 2-Pyridinecarboximidic Acid, Hydrazide

### Pyridine 2-Amidrazone Intermediate

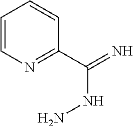

To a 1 L four neck round-bottomed flask equipped with a mechanical stirrer, a reflux condenser, and a nitrogen inlet/outlet was charged 2-pyridinecarbonitrile (50 g, 480 mmol), hydrazine hydrate (26.4 g, 528 mmol) and ethanol (25 mL). The resulting mixture was stirred at room temperature for six hours, after which time TLC showed some remaining starting material. Another 7.2 g (144 mol) portion of hydrazine hydrate was added and the mixture heated at 50° C. for three hours. The mixture was cooled in an ice bath and filtered then dried to give 40.2 g of product as a solid. The mother liquor was concentrated, triturated with hexanes and dried to give 11.6 g of additional product as a solid (51.8 g total, 381 mmol, 79%). ¹H NMR (400 MHz, CDCl₃) 8.50 (m, 1H, Ar—H), 8.01 (d, 1H, Ar—H), 7.68 (t, 1H, Ar—H), 7.28 (m, 1H, Ar—H) 5.38 (br s, 2H, —NH₂), 4.62 (br s, 1H, —N—H), 2.40 (br s, 1H, —N—H).

### Pyridine, 2-(5-(1-butylheptyl)-1,2,4-triazole-3-yl)-

### (Pyr124TzC11) Compound 16

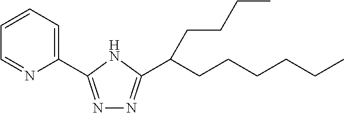 [SMILES: CCCCCCC(CCCC)c1nnc(-c2ccccn2)[nH]1]

The Pyr124TzC11 compound was synthesized using the procedure described above for the Pyr124TzVC9 compound, starting with the 2-butyloctanoyl amidrazone described below. Cydization by heating with ethylene glycol followed by automated column chromatography (220 g column, isocratic elution with 70% ethyl acetate/hexane for ten minutes, product eluted between 2-5.25 minutes) gave 8.4 g (77% yield) of the desired product, melting temperature T.=78° C. ¹H NMR (400 MHz, CDCl₃) 13.4 (br s, 1H, N—H), 8.75 (d, 1H, Ar—H), 8.23 (d, 1H, Ar—H), 7.82 (t, 1H, Ar—H) 7.38 (m, 1H, Ar—H), 2.91 (m, 1H, —CH(Bu)(Hex)), 1.82 (m, 2H, CH₂Pr), 1.72 (m, 2H, CH₂-Pent), 1.35-1.15 (m, 12H, —CH₂—), 0.82 (m, 6H, -Me)

### 2-Pyridinecarboximidic Acid, N-(2-Butyloctanoyl), Hydrazide

### (N-(2-Butyloctanoyl) Amidrazone Intermediate)

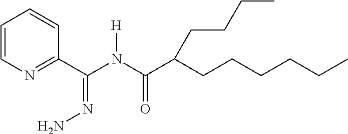 [SMILES: CCCCCCC(CCCC)C(=O)N/C(=N/N)c1ccccn1]

The N-butyloctanoyl amidrazone intermediate was prepared using the procedure described above for the branched-nonanoyl derivative. Thus, 5.3 g (38.97 mmol) of amidrazone (preparative method described above) reacted with freshly prepared 2-butyloctanoyl chloride (see preparative method below) to give the amide intermediate 11.9 g (96% yield) as a beige solid. ¹H NMR (400 MHz, DMSO-d6) 9.7 (s, 0.4H, N—H), 9.8 (s, 0.6H, N—H), 8.55 (m, 1H, Ar—H), 8.05 (m, 1H, Ar—H), 7.83 (t, 1H, Ar—H) 7.42 (m, 1H, Ar—H), 6.60 (ds, 2H, —NH₂), 3.45 (m, 0.4H, CHBu), 2.23 (m, 0.6H, CHBu), 1.65-0.75 (m, 22H, aliphatics)

### 2-Butyloctanoyl Chloride (Intermediate)

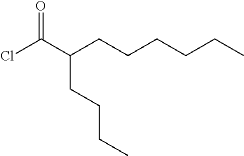

An oven dried 250 mL 3 neck round-bottomed flask was equipped with a thermocouple port, a magnetic stirrer, a condenser and a nitrogen inlet/outlet. The gas outlet was connected to a sodium hydroxide-containing scrubber (12% NaOH). The 2-butyloctanoic acid (10.0 g, 50 mmol) was combined with thionyl chloride (5.5 mL, 74.9 mmol) in CH₂Cl₂ (20 mL) and stirred at room temperature overnight. The reaction was judged to be complete by gas chromatography at that time. The reaction mixture was concentrated on the rotary evaporator then stirred under high vacuum to yield 10.8 g of material (100%). IR (neat) 1790 cm⁻¹ (C═O). No acid C═O band at 1710 cm⁻¹.

### Pyridine, 2-(5-(2,4,4-trimethylpentyl)-1,2,4-triazole-3-yl)-

### (Pyr124TzC8) Compound 17

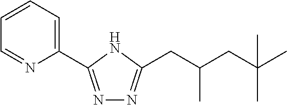 [SMILES: CC(Cc1nnc(-c2ccccn2)[nH]1)CC(C)(C)C]

The Pyr124TzC8 compound was synthesized using the procedure described above for the Pyr124TzVC9 compound, starting with the N-(3,5,5-trimethylhexanoyl) amidrazone described below. Cyclization of this intermediate (10.8 g; 38.98 mmol) in ethylene glycol (20 mL) at 180° C. over two 2 hours, followed by concentration and automated flash chromatography in two portions (120 g column, 25 minute run, gradient of from 20% to 70% ethyl acetate/hexane mixture over 12.5 min; hold at 70% for ten minutes, gradient from 70% to 100% ethyl acetate over two minutes, then 100% for 0.5 min; product eluted at from 10 min to 15 min) gave the product, 8.95 g. (89% yield) as a white solid, Tₘ=120° C. ¹H NMR (400 MHz, CDCl₃) 13.7 (br s, 1H, N—H), 8.78 (d, 1H, Ar—H), 8.25 (d, 1H, Ar—H), 7.85 (t, 1H, Ar—H) 7.39 (m, 1H, Ar—H), 2.84 (m, 1H, —CH₂(CH)Me), 2.70 (m, 1H, other CH₂(CH)Me), 2.15 (m, 1H, CHMe), 1.40 (m, 1H, CH₂-tBu), 1.15 (m, 1H, other CH₂-tBu), 1.00 (d, 3H, Me) 0.87 (s, 9H, -Me₃).

### 2-Pyridinecarboximidic Acid, N-(3,5,5-Trimethylhexanoyl), Hydrazide

### (N-(3,5,5-Trimethylhexanoyl) Amidrazone Intermediate)

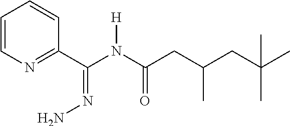 [SMILES: CC(CC(=O)N/C(=N/N)c1ccccn1)CC(C)(C)C]

To the amidrazone (synthesized previously as described above) (6 g; 44 mmol) and sodium carbonate (4.7 g; 44 mmol) was added dimethylacetamide (45 mL) and tetrahydrofuran (15 mL) under N2. The mixture was cooled to 0° C. and the trimethylhexanoyl chloride (7.8 g; 44 mmol) in DMA (15 mL) was added slowly. The mixture was stirred at room temperature for one hour. The thick mixture was concentrated, water was added and the resulting mixture filtered. The filter cake was washed with water and dried to give the product, 11.1 g, (91% yield) as a white solid. ¹H NMR (400 MHz, DMSO-d6) 9.8 (s, 0.5H, N—H), 9.7 (s, 0.5H, N—H), 8.55 (m, 1H, Ar—H), 8.05 (m, 1H, Ar—H), 7.83 (t, 1H, Ar—H) 7.42 (m, 1H, Ar—H), 6.60 (ds, 2H, —NH₂), 2.50 (m, 1H, CHMe), 2.10 (m, 2H, CH₂—CHMe), 1.30 (m, 1H, CH₂tBu), 1.10 (m, 1H, other CH₂tBu) 0.95-0.85 (m, 12H, 4Me).

### Pyridine, 2-(methyl-5-branched-nonyl-1,2,4-triazole-3-yl)- (Mixture)

### Pyridine, 2-(1-methyl-5-branched-nonyl-1,2,4-triazole-3-yl)-

### Pyridine, 2-(2-methyl-5-branched-nonyl-1,2,4-triazole-3-yl)-

### Pyridine, 2-(3-methyl-5-branched-nonyl-1,2,4-triazole-3-yl)- Mixture 18

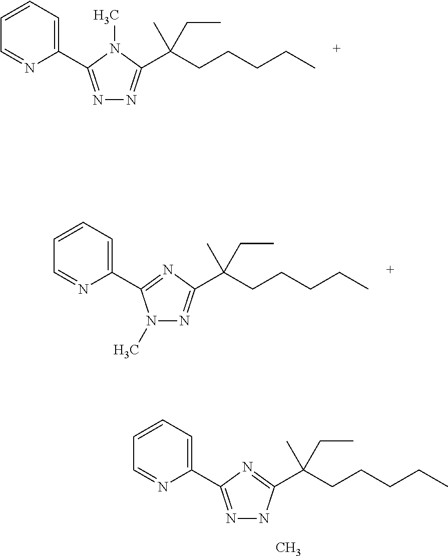

The Pyr124TzVC9 triazole (preparative method described above) (5.45 g, 0.037 mol), toluene (30 mL), iodomethane (5.8 g, 0.041 mol), and potassium carbonate (5.65 g, 0.041 mol) were sealed in a one neck, 100 mL flask. The flask was placed in a 60° C. oil bath, behind a blast shield. The reaction was incomplete after four hours of heating. Iodomethane (0.1 mol/mol, based on the amount of substance of the triazole) and potassium carbonate (0.1 mol/mol, idem) were added to the reaction and heating resumed for four hours. The reaction mixture was partitioned between ethyl acetate and water. The aqueous layer was extracted with ethyl acetate. The combined organics were washed with water then brine and dried with Na₂SO₄. The material was then adsorbed to silica and purified by automated flash chromatography (120 g column, eluted with a gradient mixture of from 0% to 20% ethyl acetate/hexanes over twelve minutes at 30 mL/min) to yield 1.66 g (28%) of the mixed methylated product. ¹H NMR (400 MHz, CDCl₃) δ 8.7 (d, 1H, Ar), 8.2 (d, 1H, Ar), 7.8 (t, 1H, Ar), 7.3 (m, 1H, Ar), 4.3 (bs, 3H, Me), 2.1-0.6 (m, 19H, aliph).

### Pyridine, 2-(octadecyltetrazole-5-yl) (mixture)

### Pyridine, 2-(1-octadecyltetrazole-5-yl) (Pyr-Ttz-1-C18) Compound 19a

### Pyridine, 2-(2-octadecyltetrazole-5-yl) (Pyr-Ttz-2-C18) Compound 19b

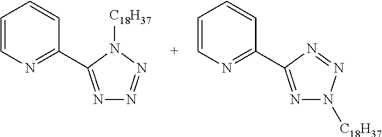

2-Pyridyl carbonitrile (12.5 g, 0.12 mol) was charged to a 2 L, three neck round bottomed flask equipped with condenser with attached N₂ inlet-outlet, mechanical stirrer and thermocouple. Solid NH₄Cl (6.4 g, 0.12 mol) was added. DMF (880 mL) then water (6.27 g) was added followed by sodium azide (7.8 g, 0.12 mol). The reaction was refluxed at approximately 137° C. overnight. A new very polar spot was seen by TLC but some starting material remained. NaN₃ and NH₄Cl, (each 0.05 mol/mol, based on the amount of substance of the 2-pyridyl carbonitrile) was added followed by a small amount of water. The reaction was complete after a total of about 65 h at 137° C. The mixture was carefully concentrated to remove DMF (CAUTION; a small amount of azide remains). The residue was dissolved in aqueous Na₂CO₃ (8 g, 0.075 mol in 0.3 L of aqueous solution). This aqueous layer was washed with ethyl acetate (2×200 mL). The aqueous layer was carefully brought to a pH of from 4 to 5 under good ventilation as traces of hydrazoic acid may form. The solid product (7 g) precipitated out of solution. The pH was then brought to between 1 and 2, and another batch of crystals was collected (3.8 g). These batches were shown to be the same substance by NMR. The resulting 2-pyridyl tetrazole (10.24 g, 0.07 mol) was allowed to react with bromooctadecane (25.2 g, 0.077 mol) and triethylamine (7.8 g, 0.077 mol) in acetonitrile (80 mL) at 97° C. for about 7.5 h. The solvent was removed under reduced pressure. The isomers were isolated by automated flash chromatography (120 g column; eluted with a gradient of from 0% to 25% ethyl acetate/hexanes over thirty minutes. The flow was 85 mL per minute.) The less polar spot had a mass of 8.3 g. The more polar spot had a mass of 13.6 g. The total yield of the reaction was 81%. HMBC NMR analysis led to the following structural assignments:

Pyr-Ttz-1-C18: Less polar spot: ¹H NMR (400 MHz, CDCl₃) δ 8.7 (dd, 1H, ArH), 8.3 (d, 1H, ArH), 7.9 (t, 1H, ArH), 7.4 (m, 1H, ArH), 5.0 (t, 2H, NCH₂), 1.9 (m, 2H, NCH₂CH₂), 1.4-1.2 (m, 28H, aliph), 0.85 (t, 3H, Me). Melting point: 67.6-68.9° C. Pyr-Ttz-2-C₁₈: More polar spot: ¹H NMR NMR (400 MHz, CDCl₃) δ 8.8 (dd, 1H, ArH), 8.3 (d, 1H, ArH), 7.8 (t, 1H, ArH), 7.4 (m, 1H, ArH), 4.7 (t, 2H, NCH₂), 2.1 (m, 2H, NCH₂CH₂), 1.4-1.2 (m, 28H, aliph), 0.9 (t, 3H, Me). Melting temperature was between 66.4° C. and 67.9° C.

### 1,3,5-Triazine, 2,4-bis(2,4-dimethylphen-1-yl)-6-1H-pyrazol-1-yl

### (DMPTriazPz) Compound 20

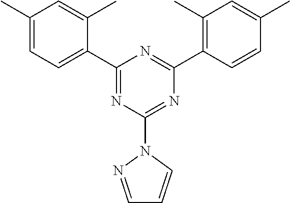

A 100 mL single-neck round-bottomed flask equipped with a magnetic stir bar and condenser was charged with 24.28 g (75 mmol) of 1-chloro-3,5-bis(2,4-dimethyl-phenyl)triazine (CDMPT) and 15.32 g (225 mmol, 3.0 eq.) of pyrazole. The mixture was heated in an oil bath at 125° C. for 5.5 h, followed by 145° C. for two hours. No CDMPT were detected by thin layer chromatography (TLC) or gas chromatography (GC). The mixture was cooled to room temperature and the resulting solid was dissolved in 300 mL of methylene chloride. The solution was washed with 3×400 mL of water, dried (molecular sieves), filtered and rotary evaporated, leaving 22.3 g of white solid after reduced pressure drying overnight at 40° C. ¹H NMR (400 MHz, CDCl₃) 8.75 (s, 1H, Pyrazole 5-H), 8.23 (d, 2H, o-Ar—H), 7.93 (s, 1H, Pyrazole 3-H) 7.15 (m, 4H, m-Ar—H), 6.55 (m, 1H, Pyrazole 4-H), 2.69 (s, 6H, o-CH₃), 2.40 (s, 6H, p-CH₃).

### 1,3,5-Triazin-2-amine, 4-(4-morpholino)-6-1H-pyrazol-1-yl-N-cyclohexyl

### (CHAMorphTriazPz) Compound 21

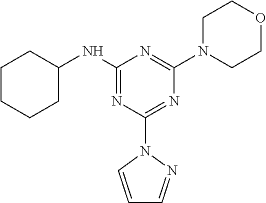 [SMILES: c1cnn(-c2nc(NC3CCCCC3)nc(N3CCOCC3)n2)c1]

A 100 mL single-neck round-bottomed flask equipped with a magnetic stir bar and condenser was charged with 10.0 g (33.6 mmol) of 1-chloro-3-cyclohexylamino-5-morpholinotriazine (CCAMT), 6.86 g (101 mmol, 3.0 mol/mol, based on the amount of substance of CCAMT) of pyrazole and 20 mL of xylenes. The mixture was heated to reflux in an oil bath for 4 hours. No unreacted CCAMT was discovered by TLC or GC. The mixture was cooled to room temperature and dissolved in 300 mL of methylene chloride. The solution was washed with 8×400 mL of water, dried (molecular sieves), filtered and rotary evaporated, leaving 9.8 g of an off-white solid after reduced pressure drying overnight at 40° C. ¹H NMR (400 MHz, CDCl₃) 8.48 (s, 1H, Pyrazole 5-H), 7.78 (s, 1H, Pyrazole 3-H), 6.42 (m, 1H, Pyrazole 4-H), 5.52 (d, 1H, —N—H—CH), 3.80 (m, 1H, —N—H—CH), 3.70-4.0 (m, 8H, morph —CH₂—), 2.1-1.15 (m, 10H, cyclohexyl —CH₂—).

### 1,3,5-Triazin-2,4-diamine-6-1H-pyrazol-1-yl-N2-branched-nonyl-N4,N4 dicyclohexyl

### (VACAPTz) Compound 22

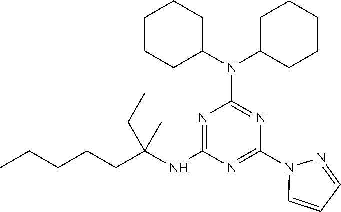 [SMILES: CCCCCC(C)(CC)Nc1nc(N(C2CCCCC2)C2CCCCC2)nc(-n2cccn2)n1]

A 100 mL single-neck round-bottomed flask equipped with a magnetic stir bar and condenser was charged with 4.5 g (10.33 mmol) of 1-chloro-3-dicyclohexylamino-5-branched-nonylaminotriazine, 2.1 g (30.99 mmol, 3.0 mol/mol, based on the amount of substance of triazine) of pyrazole and 12 mL of xylenes. The mixture was heated in an oil bath at reflux for 17 hours, GC and TLC showed incomplete reaction. Another 3.52 g (51.9 mmol, 5.0 mol/mol) of pyrazole was added and the mixture was heated for an additional eighteen hours. GC and TLC showed no remaining triazine starting product. The mixture was diluted with 50 mL of ethyl acetate and washed with 13×50 mL portions of water. The organic layer was dried over sodium sulfate, filtered and rotary evaporated, giving 2.9 g, after automated flash chromatography (220 g column, gradient of from 0% to 50% ethyl acetate hexane over fifteen minutes, product elutes at from 10 min to 13 min) (6.2 mmol, 60%), of the product as a white solid. ¹H NMR (400 MHz, CDCl₃) 8.45 (s, 1H, Pyrazole 5-H), 7.73 (s, 1H, Pyrazole 3-H), 6.41 (m, 1H, Pyrazole 4-H), 5.50-5.70 (m, 2H, —NH), 1.85-0.70 (m, 39H, aliphatics).

### 1,3,5-Triazin-2,4-diamine-6-chloro-N2-branched-nonyl-N4,N4-dicyclohexyl

### Branched-Nonylamino-Dicyclohexylamino-Chlorotriazine Intermediate

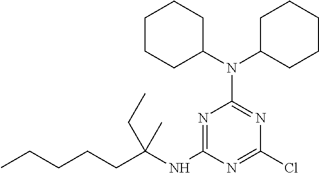 [SMILES: CCCCCC(C)(CC)Nc1nc(Cl)nc(N(C2CCCCC2)C2CCCCC2)n1]

To a 250 mL three neck round-bottomed flask fitted with two addition funnels and a mechanical stirrer was charged 5.0 g (15.19 mmol) of dichlorodicydohexylaminotriazine, 18 mL of toluene and 8 mL of water. The mixture was cooled in an ice bath and Versatic amine (preparative method described below) (2.82 g, 19.75 mmol) and 50% strength aqueous NaOH solution (1.58 mL, 19.75 mmol) were added simultaneously and dropwise over between seven and ten minutes. The mixture was stirred overnight at room temperature, after which time TLC showed only partial reaction. Heating the mixture for twenty-four hours at 68° C. gave virtually complete conversion. The mixture was cooled to room temperature, diluted with ethyl acetate, washed with water, dried over sodium sulfate, filtered, and rotary evaporated, giving 4.95 g of the product as a white solid. ¹H NMR (400 MHz, CDCl₃) 4.95 (d, 1H, —NH—CH), 2.55 (m, 1H, —CH-?), 1.85-0.70 (m, 39H, aliphatics).

### Branched-Nonylamine

### (Versatic Amine) (Intermediate)

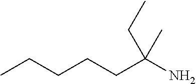

A 500 mL three neck round-bottomed flask equipped with a mechanical stirrer and addition funnel was charged with 10.0 g (52.63 mmol) of Versatic® acid chloride (preparative method described above) and 90 mL of acetone. To this solution was added dropwise 5.74 g (88.3 mmol) of sodium azide in 40 mL of water. The mixture was stirred at 0° C. for 30 minutes then poured into 120 mL of ice water. The aqueous solution was extracted with toluene (2×150 mL), dried (sodium sulfate) and gravity filtered through filter paper. (WARNING: this solution should not be concentrated, as the residue can evolve nitrogen violently). The toluene solution was then heated in an oil bath at from 60° C. to 90° C. until nitrogen evolution ceased. The GC of this material was identical to that of the acyl azide (rearrangement of the azide occurs in the injection port). The IR spectrum showed an isocyanate band (2360 cm⁻¹) and no azide band (2130 cm⁻¹). The organic solution was concentrated, giving 8.89 g of a brown oil. This material was combined directly with an 18% strength aqueous hydrochloric acid solution and then heated under reflux for three hours. GC showed disappearance of isocyanate. The mixture was cooled in an ice bath, made basic with a 10% strength aqueous solution of NaOH, and extracted with hexane, 2×200 mL. The hexane layer was washed with water and brine, dried over sodium sulfate, filtered and rotary evaporated, giving 3.01 g (40%) of the product as a brown oil. ¹H NMR (400 MHz, CDCl₃) 1.70-0.80 (m, 19H, aliphatics).

### 1,3,5-Triazin-2,4-diamine-6-1H-pyrazol-1-yl-N2,N4-bis(branched-nonyl)

### (BVAPTz) Compound 23

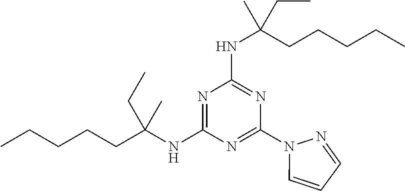 [SMILES: CCCCCC(C)(CC)Nc1nc(NC(C)(CC)CCCCC)nc(-n2cccn2)n1]

To a 100 mL single neck round bottomed flask was charged 14.25 g (36 mmol) of bis(Versatic® amino)-chlorotriazine (preparative method described below), 7.3 g (108 mmol, 3.0 mol/mol, based on the amount of substance of the triazine compound) of pyrazole, and 40 mL of xylenes. The mixture was heated to reflux for three hours, and then cooled to room temperature. No starting material remained as evidenced by TLC. The mixture was diluted with 200 mL of dichloromethane, washed with 10×250 mL of water, dried (molecular sieves), filtered and rotary evaporated, leaving 12.0 g of a paste after high vacuum treatment. NMR showed mainly product, as well as bis- and tris-pyrazole impurities. The mixture was purified using automated flash chromatography (220 g column, thirty minutes run, gradient of from 0% to 50% ethyl acetate/hexane over fifteen minutes, constant at 50% over five minutes, gradient of from 50% to 80% over two minutes, constant at 80% over eight minutes; monopyrazole eluted at from 10 min to 13 min, bis(pyrazole) eluted at from 20 min to 25 min). 8.1 g (18 mmol, 50%) of the product was thus obtained as a white solid. ¹H NMR (400 MHz, CDCl₃) 8.43 (s, 1H, Pyrazole 5-H), 7.75 (s, 1H, Pyrazole 3-H), 6.41 (m, 1H, Pyrazole 4-H), 5.60 (br d, 2H, —NH), 2.20-0.80 (m, 39H, aliphatics).

### 1,3,5-Triazin-2-amine, 4,6-di-1H-pyrazol-1-yl-N-branched-nonyl

### (VABPTz) Compound 24

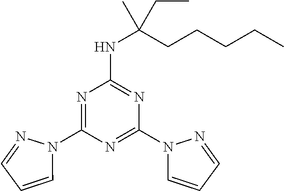 [SMILES: CCCCCC(C)(CC)Nc1nc(-n2cccn2)nc(-n2cccn2)n1]

The title compound was isolated from the reaction of a mixture of mono- and dichloro-, bis- and mono(Versatic amino) triazines (see above for reaction and separations conditions). ¹H NMR (400 MHz, CDCl₃) 8.55, 8.75 (m, 2H, Pyrazole 5-H), 7.80, 7.90 (s,s 2H, Pyrazole 3-H), 6.55, 6.50 (s,s 2H, Pyrazole 4-H), 6.0-5.80 (m, 1H, —NH), 2.20-0.80 (m, 19H, aliphatics).

### 1,3,5-Triazin-2,4-diamine-6-chloro-N2,N4-branched-nonyl

### Bis(Branched-Nonylamino)-Chlorotriazine

### Bis(Versatic Amino)Chlorotriazine

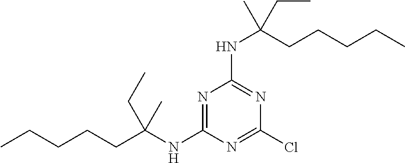 [SMILES: CCCCCC(C)(CC)Nc1nc(Cl)nc(NC(C)(CC)CCCCC)n1]

To a 250 mL three neck round-bottomed flask equipped with a stir bar, a thermocouple and two addition funnels were charged 7.3 g (39.6 mmol) of cyanuric chloride, 25 mL of toluene, and 15 mL of water. The flask was immersed in an ice bath and dual addition of a solution of NaOH (3.49 g, 87.2 mmol) in 7 mL of water, and Versatic® amine (preparative method described above) (12.5 g, 87.2 mmol) in 5 mL of toluene, was started. The addition was carried out over fifty minutes, keeping the temperature between 3° C. and 17° C. The mixture was allowed to warm to room temperature, then was diluted with 200 mL of methylene chloride, washed with 2×200 mL of water, dried (molecular sieves), filtered and rotary evaporated, leaving 14.3 g of product as an oil. The material appeared to have contained dichloro-Versatic® aminotriazine, based on the product distribution from the subsequent reaction above. ¹H NMR (400 MHz, CDCl₃) 5.25 (br d, 2H, NH) 2.05-0.75 (m, 19H, aliphatics).

### 1,3,5-Triazin-2-amine, N-methyl-N-butyl-4-1H-pyrazol-1-yl-6-(branched-decyloxy)

### (MBADoPzTz) Compound 25

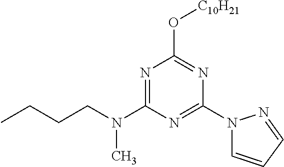

A 100 mL four neck round bottomed flask was equipped with two stoppers, a thermocouple, a magnetic stirrer and a condenser supporting a nitrogen inlet/outlet. The triazine mixture (8.8 g, 0.02 mol) was transferred to the reaction vessel using xylene (24 mL). The pyrazole (2.5 g, 0.036 mol) was then added. The reaction was refluxed (about 140° C. pot temperature) for two hours. The reaction mixture was then partitioned in between dichloromethane and water. The aqueous layer was again extracted with CH₂Cl₂. The combined organics were washed with water, then brine, and then dried with sodium sulfate. The material was concentrated under reduced pressure and purified by automated flash chromatography (120 g column with a gradient of from 0% to 20% ethyl acetate/hexanes over fourteen minutes at a flow of 150 mL/min) to yield 3.0 g (35%) of a colorless oil. ¹H NMR (400 MHz, CDCl₃) δ 8.5 (dd, 1H, pyrazole-H), 7.8 (t, 1H, pyrazole-H), 6.4 (bs, 1H, pyrazole-H), 4.4 (m, 2H, OCH₂), 3.7 (t, 1H, NCH), 3.6 (t, 1H, NCH), 3.3-3.1 (2S (rotomers, Me), 3H), 1.9-0.6 (m, 26H, aliph).

### 1,3,5-Triazin-2-amine, N-methyl-N-butyl-4-chloro-6-(branched-decyloxy)

### 2-Methylbutylamino-4-branched-decyloxy-6-chlorotriazine Intermediate

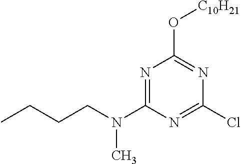

A 250 mL three neck round bottomed flask was equipped with two syringes, a thermocouple, and a nitrogen inlet/outlet. The triazine ether (preparative method described below) was transferred to the flask using toluene (15 mL). Water (10 mL) was then added. The reaction vessel was cooled to 1.5° C. using an ice bath. Sodium hydroxide (1.3 g of 50% strength aqueous solution) and N-methyl-N-butylamine (1.4 g, 0.16 mol) were added dropwise via syringe over thirty minutes. After three hours, the reaction mixture was partitioned between dichloromethane and water. The aqueous layer was again extracted with dichloromethane. The combined organics were washed with water, then dried with sodium sulfate. The material was purified by automated flash chromatography (220 g column with gradient of form 0% to 9% ethyl acetate/hexanes over twenty-five minutes at a flow of 150 mL/min) to yield 8.8 g of product. LC/MS showed the desired product along with an undetermined amount of triether triazine. ¹H NMR (400 MHz, CDCl₃) δ 4.4-4.3 (m, 2H, OCH₂), 3.6 (q, 2H, NCH₂), 3.2-3.1 (2s (rotomers), CH₃N), 1.9-0.6 (m, 26H, aliph).

### 1,3,5-Triazine, 2,4-dichloro-6-(branched-decyloxy)

### 2-branched-Decyloxy-4,6-dichlorotriazine Intermediate

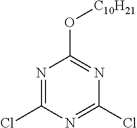

To a 500 mL three neck round-bottomed flask equipped with a stir bar, a thermocouple and two addition funnels was charged 25.0 g (136 mmol) of cyanuric chloride, 100 mL of toluene, and 65 mL of water. The flask was immersed in an ice bath and dual addition of caustic (5.44 g, 136 mmol) in 5.44 mL of water, and Exxal® 10 alcohol (a commercial mixture of isomeric aliphatic alcohols having from nine to eleven carbon atoms, with the majority being C₁₀ alcohols, sold by Exxon Mobil Corporation of Irving, Tex. USA, 21.5 g, 136 mmol), was started. The addition was carried out over thirty minutes, keeping the temperature between 6° C. and 10° C. The mixture was allowed to warm to room temperature, then was poured into 500 mL of water. The resulting mixture was extracted with 2×250 mL of dichloromethane and 8 g of a white solid was filtered off. The organic filtrate was dried (sieves), filtered and rotary evaporated, leaving 29 g of product as a milky oil. The material was a mixture of the mono-, bis-, and tris(decyloxy) triazines (mainly bis) by NMR and by analysis of the subsequent reaction mixtures above. ¹H NMR (400 MHz, CDCl₃) 5.25 (br d, 2H, NH) 2.05-0.75 (m, 19H, aliphatics).

### 10,12,13,15-Tetracosanetetraone Intermediate

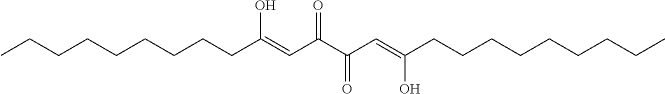 [SMILES: CCCCCCCCC/C(O)=C/C(=O)C(=O)/C=C(\O)CCCCCCCCC]

Under nitrogen, 2-undecanone (8.7 g, 0.051 mol) was added slowly to 1.67 g of a slurry of sodium hydride (having a mass fraction of sodium hydride in the mineral oil slurry of 60%, 0.042 mol) and dry toluene (50 mL) at 60° C. followed by diethyl oxalate (3.45 mL, 0.025 mol). The reactants were mixed at 60° C. for eight 8 hours, cooled to room temperature, then poured into a slurry of ice water and acetone (ca. 200 mL) and extracted with ethyl acetate (3×50 mL). The combined organic phases were dried over sodium sulfate and filtered under reduced pressure. Ethyl acetate was removed by distillation under reduced pressure, and the crude solid residue was recrystallized from methanol to give 10,12,13,15-tetracosanetetraone as a pale yellow solid (7.01 g, 70%). ¹H NMR (500 MHz): 0.91 (6H, t), 1.34 (24H, m), 1.68 (4H, q), 2.50 (4H, t), 6.36 (2H, s)

### 5,5′-nonyl-3,3′-bi-1H-pyrazole Compound 26

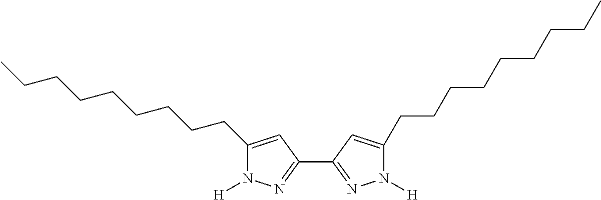 [SMILES: CCCCCCCCCc1cc(-c2cc(CCCCCCCCC)[nH]n2)n[nH]1]

10,12,13,15-Tetracosanetetraone (1.50 g, 0.004 mol) and hydrazine hydrate (0.40 g, 0.008 mol) were combined in ethanol at 80° C. and heated to reflux for four hours. Ethanol was removed under reduced pressure, and the residue was partially dissolved in ethyl acetate (10 mL) and deionised water (10 mL), then both phases were filtered together under reduced pressure to collect 5,5′-nonyl-3,3′-bis-1H-pyrazole as a yellow solid (1.03 g, 71%).

¹H NMR (500 MHz): 0.90 (6H, t), 1.30 (24H, m), 1.72 (4H, q), 2.72 (4H, t), 6.36 (2H, s) Mass Spectrum (m/z): 387.35 (Mt), 409.33 (M⁺+Na⁺)

### Example 1

### Selectivity Testing

Aqueous solutions PLS1 and PLS2 (modeling pregnant leach solutions) have been prepared by dissolving reagent grade salts as listed in Table a, wherein the concentrations of the various metal ions in PLS1 and PLS2 are given. pH was adjusted to 3 (for PLS1) and 1 (for PLS2) by addition of sulfuric acid. Mass concentrations γ_{M} of the metals in these solutions are stated in Table a in mg/L, where γ_{M}=m_{M}/V_{S}, V_{S} being the volume of the solution, and m_{M} being the mass of the metal.

Organic phases were prepared according to the concentrations shown in Table b, which provides a summary of the selectivities of each extractant for Ni, Co, and various other metal ions. Aliquots of the organic phase (5.5 mL) were mixed with portions of one of the PLS compositions in Table a (5.0 mL) and 0.5 mL of H₂SO₄ solution, water or NaOH solution to obtain a range of final pH values after stirring for at least two hours. The phases were then separated and filtered (the organic phases through Whatman 1PS phase separating paper and the aqueous phases through Whatman 1 filter paper) and the metal content of each was analysed. The pH0.5 (the value of pH where the metal ions under consideration are equally distributed between aqueous and organic phases) was measured from the resulting plot of extraction vs. pH.

TABLE a
Composition of Synthetic Ni Laterite PLS (mass concentrations
γ_{M} of metals in the solutions denoted as “PLS1”
and “PLS2”, in mg/L)

| Element | PLS1 | PLS2 | Source |
|---|---|---|---|
| Ni | 5000 | 5000 | NiSO₄•6 H₂O |
| Co | 500 | 500 | CoSO₄•7 H₂O |
| Mn | 1500 | 2900 | MnSO₄•H₂O |
| Mg | 15000 | 15000 | MgSO₄ |
| Zn | 100 | 200* | ZnSO₄•7 H₂O |
| Ca | 500 | 0 | CaCl₂ |
| Na | 2800 | 0 | NaCl |
| Fe | 20 | 3200 | Fe₂(SO₄)₃•7 H₂O |
| Si | 15 | 0 | Na₃SiO₄H (solution) |
| Al | 0 | 200 | Al₂(SO₄)₃•16 H₂O |
| Cr | 0 | 100 | Cr₂(SO₄)₃•xH₂O |
| Cu | 200 | 200* | CuSO₄•5 H₂O |
| pH | 3 | 1 | H₂SO₄ |

*not always present

TABLE b
Selectivity Test results for different extractants

| Extraction System | | | |
|---|---|---|---|
| c(E) = 0.28 mol/L | pH_{0.5} | | Other Metals Extracted >5% |
| c(DNNSA) = 0.185 mol/L | Ni | Co | in S-Curve (pH range) |
| only DNNSA, c = 0.4 mol/L | >1.3 | >1.3 | Mn, Mg, Fe; (0.5-1.3) |
| Comp. 1 (PyrPzVC9) + DNNSA | <0 | 0.75 | N/A; (0-1.7) |
| Comp. 2 (PyrPzC9n) + DNNSA | N/A | N/A | N/A |
| Comp. 3 (PyrNPzH) + DNNSA (4) | <<0 | <0 | Al, Cr; (0.1-1.4) |
| Comp. 4 (Pyrbis(PzC9n)) + DNNSA | NS | NS | NS |
| Comp. 5 (PyzPzVC9) + DNNSA | <<0.5 | <0.5 | N/A; (0.6-1.6) |
| Comp. 6 (PyrIsoxVC9A) + DNNSA | >1.5 | >1.5 | Mn, Mg; (0-1.3) |
| Comp. 7 (PyrIsoxVC9B) + DNNSA | >2 | >2 | Mn, Mg, Fe; (0-1.9) |
| Comp. 8 (Pyr124OdC11) + DNNSA | <<1.3 | 2 | Zn, Cu**; (1.2-2.7) |
| Comp. 9 (Pyr124OdC8) + DNNSA | <0 | 1.2 | Cu, Zn, Mn**; (0-2.1) |
| Comp. 10 (Pyr124OdVC9) + DNNSA | 1.5 | >2 | Cu, Zn, Mn**; (0.6-1.8) |
| Comp. 11 (Pyr124OdC17NissN) + DNNSA | <0 | 1.2 | Cu, Zn**; (0-1.7) |
| Comp. 12 (Pyr124OdC17soy) + DNNSA | <1.3^{#} | >1.3^{#} | Cu, Zn^{#}**; (1.3) |
| Comp. 13 (Pyr134OdVC9) + DNNSA | 0 | 2 | Cu, Zn**; (0-2.0) |
| Comp. 14 (Pyr134OdC15) + DNNSA | <1.3^{#} | >1.3^{#} | Cu, Zn^{#}**; (1.3) |
| Comp. 15 (Pyr124TzVC9) + DNNSA | <<0 | 1.3 | N/A, (0-1.6) |
| Comp. 16 (Pyr124TzC11) + DNNSA | N/A | N/A | Formed Precipitate |
| Comp. 17 (Pyr124TzC8) + DNNSA | N/A | N/A | Formed Precipitate |
| Mix. 18 (Pyr124TzMeVC9) + DNNSA | >2.1 | >2.1 | Cu, Zn, Al, Cr, Mg, Mn**; (0.8-2.1) |
| Comp. 19a (PyrTtz1C18) + DNNSA (4) | 0.1 | 1.2 | N/A; (0-1.50) |
| Comp. 19b (PyrTtz2C18) + DNNSA | <0 | >2 | N/A; (0-2.0) |
| Comp. 20 (DMPTriazPz) + DNNSA (4) | <0 | 0.2 | Mg; (0-1.7) |
| Comp. 21 (CHAMorphTriazPz) + DNNSA (4) | <0 | 0.6 | N/A; (0-1.2) |
| Comp. 22 (VACAPTz) + DNNSA | <1.3^{#} | <1.3^{#} | Cu, Zn, Mn^{#}**; (1.3) |
| Comp. 23 (BVAPTz) + DNNSA | <1.6^{#} | <1.6^{#} | Cu, Zn^{#}**; (1.6) |
| Comp. 24 (VABPTz) + DNNSA | <1.3^{#} | >1.3^{#} | Cu, Zn, Ca^{#}**; (1.3) |
| Comp. 25 (MBADoPzTz) + DNNSA | <1.2^{#} | >1.2^{#} | Cu^{#}**; (1.2) |
| (bipy)* + DNNSA (t) | <0.7 | >1.5 | Fe; (0.7-1.5) |
| terpy* + DNNSA (t) | <0.8 | >1.6 | Fe; (0.8-1.6) |
| Pyr-bis-isoxazoline* + DNNSA (t) | >2 | >2 | Fe; (0.8-2.0) |
| Pyr-isoxazoline* + DNNSA (t) | >2 | >2 | Fe; (0.8-2.0) |

NS = not soluble;
^{#}= single extraction point at specified pH;
**Cu and Zn were present in the PLS;
(4) = test was conducted in 1,1,2,2-tetrachloroethane;
(t) = test was conducted in toluene
pH_{0.5}: the value of pH where the metal ions under consideration are equally distributed between aqueous and organic phases;
DNNSA = dinonyl naphthalene sulfonic acid purchases as NACURE ® 1052 from King Industries of Norwalk, CT USA;
N/A = no other metal found;
Unless TCE or toluene are used as solvent, ORFOM ® SX-12 has been used;
*The following compounds were purchased from Sigma-Aldrich: bipy = 2,2′-bipyridyl (CAS 366-18-7) terpy = 2,2′: 6′,2″-Terpyridine (CAS 1148-79-4) Pyr-bis-isoxazoline = 2,6-Bis[(4R)-(+)-isopropyl-2-oxazolin-2-yl]pyridine (CAS 131864-67-0); and Pyr-isoxazoline = (S)-4-tert-Butyl-2-(2-pyridyl)oxazoline (CAS 117408-98-7).

### Example 2

### Extraction Equilibria

The extraction equilibria for Pyr124OdC11 (Compound 8) in combination with DNNSA were determined by mixing 5.5 mL of organic solution with 5.0 mL of PLS1 and 0.5 mL of H₂O or NaOH solution to obtain raffinate pH values between 1 and 3. The DNNSA was Nacure® 1052 supplied by King Industries of Norwalk, Conn. USA as a solution in an aliphatic solvent with a mass fraction of 50%. The organic phase contained Pyr124OdC11 (Compound 8, 0.28 mol/L) and DNNSA (0.19 mol/L) in Orfom® SX-12. Samples were magnetically stirred in 4 dram vials at 23° C. for four hours. The phases were then separated and analyzed for metal content. Table c shows the metal content for various metals of the organic phase at various pH's for a typical extractant. The extraction composition is selective for Ni, Cu, Co and Zn against Mn, Mg, Fe, Na and Ca.

TABLE c
Mass Concentration of Metals in the Organic Phase (mg/L)

| pH | Ni | Co | Cu | Zn | Mn | Mg | Fe | Na | Ca |
|---|---|---|---|---|---|---|---|---|---|
| 1.24 | 3756 | 199 | 147 | 19 | 31 | 34 | 0.0 | 15 | 0.7 |
| 1.41 | 3907 | 229 | 158 | 24 | 38 | 40 | 0.0 | 21 | 1.0 |
| 1.50 | 4004 | 257 | 162 | 27 | 47 | 47 | 0.2 | 25 | 1.4 |
| 1.68 | 3913 | 248 | 156 | 26 | 45 | 42 | 0.0 | 27 | 1.1 |
| 1.98 | 3936 | 276 | 161 | 30 | 56 | 48 | 0.0 | 33 | 1.3 |
| 2.73 | 4206 | 324 | 171 | 39 | 78 | 60 | 0.8 | 44 | 1.8 |

### Example 3

### Separation of Co and Ni from PLS1 According to the Compositions of Table a

Five cycles of extract-wash-strip-strip-wash were carried out in the extraction vessel as follows using an organic phase containing 0.28 mol/L of Pyr124OdC11 (Compound 8) and 0.19 mol/L of DNNSA in Orfom® SX-12:

The vessel consists of a jacketed, cylindrical, flat-bottomed flask of internal diameter 10 cm and depth 14 cm. It is equipped with a tap so that liquids can be run off from the bottom. It also has a ground glass flange to take a suitable lid with inlet ports to accommodate a stirrer, thermometer and sample addition. The vessel is further equipped with a removable stainless steel baffle comprising four vertical plates 10 mm wide and equidistant from each other. The vessel is water-jacketed, water being pumped from a thermostatically-controlled bath by a suitable small pump. On the outside of the jacket is a centimeter scale with zero equivalent to the bottom of the vessel. The impeller has a diameter of 5 cm, with six blades regularly spaced beneath a circular disc and two spoiler blades fixed to the upper surface. The impeller is mounted so that the bottom of the blades is 3 cm from the bottom of the vessel; it is held in position by a PTFE gland in the vessel lid. It is driven at the required speed by a high-torque variable-speed stirrer motor (variable between 600 min⁻¹ and 2200 min⁻¹).

The extraction was run at 50° C. with the agitator at 1270 min⁻¹ (1270 rpm). Two consecutive stripping stages were used, with fresh aqueous hydrochloric acid solution (2 mol/L) each time. The results for the metal mass concentrations observed are compiled in Table d, which provides the numerical values of the pH of the raffinate for each of the cycles; for a multiple load/strip, cycles with the mass concentrations of the metals in the organic phase are shown for each step.

TABLE d
Metal in organic over multiple cycles; mass concentration in
mg/L (= ppm)

| Stage | Ni | Co | Cu | Mg | Fe | Mn | Zn | Ca | pH |
|---|---|---|---|---|---|---|---|---|---|
| LO₁ | 3569 | 258 | 148 | 83 | 0 | 70 | 31 | 2 | 1.25 |
| Wash | 3593 | 265 | 149 | 114 | 10 | 89 | 43 | 8 | 2.60 |
| BO₁ | 1298 | 12 | 99 | 0 | 6 | 0 | 2 | 0 | N/A |
| BO₁ₐ | 582 | 0 | 73 | 0 | 4 | 0 | 0.4 | 0 | N/A |
| Wash | 582 | 0.2 | 61 | 0.5 | 42 | 0.1 | 1 | 1 | 1.56 |
| LO₂ | 4151 | 131 | 183 | 22 | 2 | 18 | 12 | 0.3 | 1.37 |
| Wash | 4312 | 143 | 184 | 33 | 3 | 30 | 15 | 1 | 2.63 |
| BO₂ | 1634 | 6 | 122 | 0 | 1 | 0 | 1 | 0.1 | N/A |
| BO₂ₐ | 619 | 0 | 81 | 0 | 2 | 0 | 0 | 0 | N/A |
| Wash | 649 | 0 | 73 | 0 | 106 | 1 | 0 | 1 | 1.64 |
| LO₃ | 4175 | 151 | 191 | 27 | 4 | 24 | 15 | 1 | 1.35 |
| Wash | 4342 | 179 | 209 | 45 | 8 | 41 | 19 | 2 | 2.46 |
| BO₃ | 1539 | 7 | 130 | 0 | 1.0 | 0 | 1 | 0 | N/A |
| BO₃ₐ | 578 | 0 | 85 | 0 | 7 | 0 | 1 | 0.3 | N/A |
| Wash | 425 | 11 | 71 | 13 | 38 | 8 | 19 | 10 | 1.84 |
| LO₄ | 3395 | 124 | 169 | 30 | 0.4 | 25 | 16 | 1 | 1.25 |
| Wash | 3449 | 136 | 170 | 49 | 1 | 41 | 19 | 2 | 2.27 |
| BO₄ | 1218 | 8 | 109 | 0.2 | 1 | 0 | 2 | 0.4 | N/A |
| BO₄ₐ | 550 | 0.4 | 78 | 0.1 | 4 | 0 | 1 | 1 | N/A |
| Wash | 610 | 2 | 76 | 1.0 | 67 | 1 | 1 | 1 | 1.08 |
| LO₅ | 3515 | 113 | 186 | 26 | 7 | 21 | 14 | 2 | 1.37 |
| Wash | 3696 | 130 | 193 | 33 | 12 | 30 | 16 | 2 | 2.45 |
| BO₅ | 1319 | 6 | 131 | 0.1 | 2 | 0 | 2 | 1 | N/A |
| BO₅ₐ | 597 | 0 | 91 | 0 | 7 | 0 | 1 | 1 | N/A |

LOx = loaded organic (cycle), BOx = barren organic (cycle), where x is 1 for the first cycle, 2 for the second cycle, etc., and 5 for the fifth cycle

### Example 4

### Stripping of Residual Cu with Ammonia

A washed barren organic solution containing 0.28 mol/L of Pyr124OdC11 (Compound 8) and 0.19 mol/L of DNNSA in Orfom® SX-12 was generated by extraction from PLS1, washed with water, stripped twice with 2 M HCl and then washed with water. The organic solution contained 700 mg/L of Ni and 97 mg/L of Cu. After mixing it with an ammoniacal ammonia solution (prepared from aqueous ammonia and ammonium carbonate, containing 290 g/L of NH₃ and 220 g/L of CO₂), the organic phase contained 172 mg/L of Ni and 21 mg/L of Cu, representing 75% of Ni stripping and 78% of Cu stripping.

As used herein, the terms “a” and “an” do not denote a limitation of quantity, but rather the presence of at least one of the referenced items. Recitation of ranges of values are merely intended to serve as a shorthand method of referring individually to each separate value falling within the range, and each separate value is incorporated into this specification as if it were individually recited. Thus each range disclosed herein constitutes a disclosure of any sub-range falling within the disclosed range. Disclosure of a narrower range or more specific group in addition to a broader range or larger group is not a disclaimer of the broader range or larger group. All ranges disclosed herein are inclusive of the endpoints, and the endpoints are independently combinable with each other. “Comprises” as used herein includes embodiments “consisting essentially of” or “consisting of” the listed elements.

Although the foregoing description has shown, described, and pointed out the fundamental novel features of certain embodiments of the present invention, it will be understood that various omissions, substitutions, and changes in the form of the detail of the invention as described may be made by those skilled in the art, without departing from the spirit and scope of the present teachings. Consequently, the scope of the present invention should not be limited to the foregoing description or discussion.

We claim:

1. An aliphatic-aromatic heterocyclic compound A of formula I, or salts or tautomers thereof, which comprises two rings B and C, each ring being independently five- or six-membered, and each ring having at least one nitrogen atom as heteroatom in the ring structure, wherein at least one of the rings B and C has at least one further heteroatom selected from the group consisting of nitrogen atoms and of oxygen atoms, and wherein at least two of the heteroatoms in at least one of the rings having two or more heteroatoms are directly attached to each other by a chemical bond, and wherein at least one of the rings B and C bears a further substituent R at one or more substitutable position which substituent R has from six to twenty-five carbon atoms which are optionally substituted, and wherein from 1 to 3 carbon atoms is optionally substituted with a nitrogen or oxygen atom 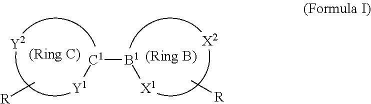
wherein in ring B, there is one heteroatom X1 which is directly bonded to ring atom B1 in ring B, and B1 is selected from the group consisting of a carbon atom and a nitrogen atom, and in ring C, there is one heteroatom Y1 which is directly bonded to ring atom C1 in ring C, and C1 is a carbon atom, and atoms B1 and C1 are connected to each other by a chemical bond which may be a single bond or a double bond, and wherein X2 and Y2 are each separately a sequence of from three atoms, in the case of a five-membered ring, to four atoms in the case of a six-membered ring, and are selected from heteroatoms and carbon atoms.

2. The aliphatic-aromatic heterocyclic compound A of claim 1 wherein ring B has five ring atoms, and ring C has five or six ring atoms.

3. The aliphatic-aromatic heterocyclic compound A of claim 1, wherein the substituent R is selected from the group consisting of a linear alkyl group, a branched alkyl group, a cyclic alkyl group, and an alkyl-substituted aryl group.

4. The aliphatic-aromatic heterocyclic compound A of claim 1, wherein the ring B is a five-membered ring having two heteroatoms whereof one is a nitrogen atom, and one further heteroatom selected from the group consisting of nitrogen and oxygen atoms, which two heteroatoms are directly attached to each other by a chemical bond.

5. The aliphatic-aromatic heterocyclic compound A of claim 1, wherein the ring B is a five-membered ring having three heteroatoms whereof two are nitrogen atoms, and one further heteroatom selected from the group consisting of nitrogen atoms and of oxygen atoms.

6. The aliphatic-aromatic heterocyclic compound A of claim 1, wherein the ring B is a five-membered ring having four heteroatoms whereof all four are nitrogen atoms.

7. The aliphatic-aromatic heterocyclic compound A of claim 4, wherein the ring C has five or six atoms in the heterocyclic ring which is either an aromatic ring, or a cycloaliphatic ring having at least one double carbon-carbon bond in the cycloaliphatic ring, and has a nitrogen atom in the 2-position relative to the atom connected to the ring B.

8. The aliphatic-aromatic heterocyclic compound A of claim 4, wherein the ring B carries a substituent R which is linear or branched or cyclic alkyl group having from six to twenty-five carbon atoms.

9. The aliphatic-aromatic heterocyclic compound A of claim 7 wherein the ring C is a pyrrole ring, a pyrazole ring, a triazole ring, a 1,3,5-triazine ring, a pyrazine ring, a pyrimidine, a pyridazine or a pyridine ring.

10. The aliphatic-aromatic heterocyclic compound A of claim 1, wherein the ring B is selected from the group consisting of pyrazole, isoxazole, 1,3,4-oxadiazole, 1,2,4-oxadiazole, 1,2,4-triazole, 1,2,3-triazole, and 1,2,3,4-tetrazole.

11. The aliphatic-aromatic heterocyclic compound A of claim 8, wherein the ring C carries substituent R at one or more substitutable positions on the ring which is chosen from i) a linear or branched alkyl or alkenyl having from six to twenty-five carbon atoms wherein from one to three carbon atoms are optionally substituted with a nitrogen or oxygen atom; or ii) a cyclohexyl wherein from one to three carbon atoms are optionally substituted with a nitrogen or oxygen atom; N-cyclohexyl; N,N-dicylcohexyl; pyrazole; or phenyl, any of which is optionally substituted with a linear or branched alkyl having from one to six carbon atoms.

12. A metal extractant composition E comprising an aliphatic-aromatic heterocyclic compound A as defined by claim 1, and an organic acid D having at least one carboxylic, sulfonic, sulfuric, phosphinic, phosphonic, or phosphoric acid group, or salt thereof.

13. The metal extractant composition E of claim 12 wherein the organic acid D has at least one carboxylic, sulfonic, sulfuric, phosphinic, phosphonic, or phosphoric acid group attached to an aromatic molecule.

14. The metal extractant composition E of claim 12 wherein the organic acid D has at least one linear, branched, or cyclic alkyl substituent having from six to twenty-five carbon atoms on the aromatic molecule.

15. The metal extractant composition E according to claim 12, wherein the amount of substance-ratio of A and D is from 95 mol:5 mol to 5 mol:95 mol.

16. The metal extractant composition E according to claim 12, wherein the mixture of the heterocyclic compound A and the organic acid D is dissolved in an organic solvent S which is not homogeneously miscible with water.

17. The metal extractant composition E as claimed in claim 16, wherein the organic solvent S is selected from the group consisting of aliphatic hydrocarbons, and mixed aromatic-aliphatic hydrocarbons.

18. A process to extract one or more metals M selected from the group consisting of Ni and Co from an aqueous acidic leach solution PL comprising ions of at least one of the metals M, and further, at least one kind of further ions selected from the group consisting of Fe ions, Al ions, Cu ions, Mg ions, Mn ions, and also silicate anions, by
mixing the solution PL with an extractant composition E as defined by claim 12, until the following condition is fulfilled for the concentration c₁(M) of metal M in the aqueous phase after mixing during a time t1: [c₀(M)−c₁(M)]/[c₀(M)−cₑ(M)]>0.1, where c₀(M) is the concentration of metal M in the solution PL before mixing, and cₑ(M) is the equilibrium concentration of metal M in the aqueous phase after mixing and reaching the equilibrium,
separating the organic and aqueous phases, and
recovering the metal M from the separated organic phase by re-extraction with an aqueous phase which is neutral, acidic or alkaline, or by precipitation of the metal M by addition of a chemical compound that forms an insoluble compound with the said metal M.
